(19) United States
(12) Patent Application Publication
(10) Pub. No.: US 2026/0306648 A1
(43) Pub. Date: Oct. 8, 2026

(54) DELIVERY OF DUAL PAYLOAD OF MMAF OR MMAE AND TLR7 AGONISTS

(71) Applicant: The Research Foundation for the State University of New York, Albany, NY (US)

(72) Inventors: Lawrence Nathan TUMEY, Vestal, NY (US); Victor OJO, Binghamton, NY (US)

(73) Assignee: The Research Foundation for the State University of New York, Albany, NY (US)

(21) Appl. No.: 19/484,461

(22) PCT Filed: May 15, 2024
(86) PCT No.: PCT/US2024/029346

§ 371 Date: Nov. 14, 2025

Related U.S. Application Data
(60) Provisional application No. 63/502,176, filed on May 15, 2023.

(51) Int. Cl. A61K 47/68; A61P 35/00

(52) U.S. Cl. CPC A61K 47/6803; A61K 47/68031; A61K 47/6851; A61K 47/6855; A61K 47/6889; A61P 35/00

(57) ABSTRACT

Disclosed herein are antibody-drug-conjugates (ADC) of Formula (A) containing both a toll-like receptor (TLR) agonist (immune stimulant) and MMAF or MMAE:

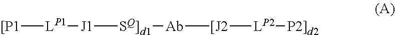

The antibody-drug-conjugates are useful as therapeutic agents for treating various cancers. Pharmaceutical compositions which include the ADCs are also disclosed. Also disclosed are methods of treating a tumor or abnormal cell proliferation by administering a therapeutically effective amount of an antibody-drug-conjugate disclosed herein under conditions effective to treat a tumor or abnormal cell proliferation. Also disclosed are methods for stimulating an immune response and/or inducing an anti-tumor immune response in a subject by administering a therapeutically effective amount of an antibody-drug-conjugate disclosed herein.

### CROSS-REFERENCE TO RELATED APPLICATION

This application claims priority of U.S. provisional application 63/502,176, filed May 15, 2023, the entire disclosure of which is hereby incorporated herein by reference.

### GOVERNMENT RIGHTS STATEMENT

This invention was made with government support under R01 GM140026 and R01 GM144450 awarded by National Institutes of Health. The government has certain rights in the invention.

### FIELD OF THE INVENTION

The invention relates to antibody-drug-conjugates (ADC) containing both a toll-like receptor (TLR) agonist (immune stimulant) and MMAF or MMAE. The targeted delivery of agonists of toll-like receptors (TLRs) and MMAF or MMAE with a single tumor-targeting antibody allows for delivery and release of the TLR 7 and/or TLR8 agonists into desired tissues, resulting in immunoactivation, while co-delivering MMAF or MMAE causes release upon internalization into the tumor cells, thereby killing the antigen-expressing cell. Compounds of the present invention are thus useful as therapeutic agents for treating various cancers.

### BACKGROUND OF THE INVENTION

Antibody-drug conjugates (ADCs) are antibodies loaded with drug payloads that allow for the delivery of those drugs to a desired cell type. The specificity of antibodies to a specific cell type results in more targeted delivery of the drugs with fewer negative side effects due to the delivery of the often cytotoxic drugs to healthy cells.

MMAE (Monomethyl auristatin E) and MMAF (Monomethyl auristatin F) are antimitotic and anti-tubulin compounds that are utilized as anticancer compounds. These compounds currently are only administered when linked to monoclonal antibodies, due to their high toxicity.

Toll-like receptors (TLRs) govern the innate immune system response through recognition of pathogen-associated molecular patterns (PAMPs). TLR7 and TLR8 are among the known human TLR endosomal receptors and are able to induce an innate immune system response and can be activated using agonists.

TLR7 and TLR8 are homologous receptors that bind and are activated by single-stranded RNA from endocytosed bacteria and viruses. Activation initiates a downstream inflammatory response, followed by creation of a complex that initiates a signaling cascade and eventually activates transcription factors such as nuclear factor kappa-light-chain-enhancer of activated B cells (NF-κB) and interferon regulatory factor 7 (IRF7). These then stimulate inflammatory cytokine and type I interferon production, which are important to many inflammatory processes. Due to differences in TLR7 and TLR8 cytokine induction profiles as well as receptor expression variability between immune cell types, activation of TLR7 or TLR8 results in unique immune responses. Likewise, secretion of cytokines from TLR7/8 activation contributes to the activation of antigen-specific T and B cells, which helps initiate the adaptive immune response. TLRs are associated with numerous immune and inflammatory conditions, and, accordingly, the ability to modulate TLR activity is a potential pathway for treatment of those conditions.

TLR agonists are immunostimulants that are often used as vaccine adjuvants (see, for instance, McGowan, D., Current Topics in Medicinal Chemistry 19:2228-2238 (2019)). These agonists activate the adaptive immune system, thus leading to a more robust anti-viral effect. TLR agonists are also being explored as a way to “unmask” the immunosuppressive tumor environment in hopes that the immune system will recognize cancer tissue as “foreign” and thus initiate a robust anti-tumor response by the immune system. TLR agonist development is fraught with inflammation-associated side effects, as is the case with commercially available TLR7 and TLR 8 agonists (see Kieffer et al., Expert Opinion on Therapeutic Patents 30(11):825-845 (2020)).

One commercially available TLR7 agonist is imiquimod. Imiquimod application is limited to topical administration due to safety concerns with system dosing. TLR agonists, such as imiquimod, if delivered systemically, result in whole-body immunostimulation, leading to acute toxicity from a cytokine-storm type of event. It is an ongoing problem to find methods for safe delivery of TLR7 and TLR8 agonists while maintaining therapeutic efficacy. Accordingly, there are numerous ongoing studies dedicated to the development of an improved delivery platform for TLR7 and TLR8 agonists that enable a robust local delivery without a systemic exposure. The development of resiquimod, motolimod, and other TLR7 and TLR8 agonists as immunostimulatory agents for use in cancer patients has faced difficulties and appears to stand at an impasse, at least in part due to disappointing results obtained in recent clinical testing (Frega et al., Oncoimmunology 9:1-10 (2020)). A major challenge in this field is development of efficacious molecules with adequate safety margins, as TLR agonists activate the innate immune system to elevate the body's inflammatory response (Kieffer et al., Expert Opinion on Therapeutic Patents 30(11):825-845 (2020); Patel et al., Future Virol. 9(9):811-829 (2014); and Tisoncik et al., Microbiol. Mol. Biol. Rev. 76(1):16-32 (2012)). Accordingly, the use of TLR agonists in immuno-oncology is an area of great interest, but there remains a significant need for improved delivery and use of TLR7 and TLR8 agonists.

The present disclosure is directed to overcoming these and other deficiencies in the art.

### SUMMARY OF THE INVENTION

Briefly, the present invention provides dual-payload antibody-drug conjugates (ADCs). These dual-payload ADCs allow for the targeted delivery of TLR agonist immune stimulators and cytotoxic MMAE/MMAF to specific tumor cells, reducing toxicity and enhancing efficacy against these cells.

The present invention provides, in a first aspect, an antibody-drug conjugate of Formula (A):

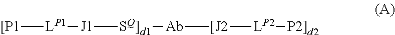

wherein:
P1 comprises a TLR7 agonist, MMAE, or MMAF;
L^{P1} comprises a cleavable or noncleavable linker;
J1 comprises a first conjugation handle;
S^{Q} is —S(C₁-C₆)alkylNH—, wherein the NH is attached to the carbonyl of a Q295 residue of Ab;
Ab comprises an antibody;
J2 comprises a second conjugation handle attached at a sulfur atom of a cysteine residue of Ab;
L^{P2} comprises a cleavable or noncleavable linker; and
P2 comprises a TLR7 agonist, MMAE or MMAF;
wherein:
d1 is between 1 and about 2;
d2 is between about 2 and about 10;
L^{P1} and L^{P2} may be the same or different;
J1 and J2 may be the same or different; and
when P1 is a TLR7 agonist, P2 is selected from MMAE or MMAF; and when P1 is MMAE or MMAF, P2 is a TLR7 agonist.

The present invention provides, in a second aspect, a pharmaceutical composition comprising an antibody-drug conjugate disclosed herein and a pharmaceutically acceptable carrier, diluent, or excipient.

The present invention provides, in a third aspect, a method for stimulating an immune response in a subject. The method includes administering a therapeutically effective amount of an antibody-drug conjugate described herein under conditions effective to stimulate an immune response.

The present invention provides, in a fourth aspect, a method for inducing an anti-tumor immune response in a subject. The method includes administering a therapeutically effective amount of an antibody-drug conjugate described herein under conditions effective to induce an anti-tumor immune response.

The present invention provides, in a fifth aspect, a method for treating a tumor or abnormal cell proliferation in a subject. The method includes administering a therapeutically effective amount of an antibody-drug conjugate described herein under conditions effective to treat a tumor or abnormal cell proliferation.

These, and other objects, features and advantages of this invention will become apparent from the following detailed description of the various aspects of the invention taken in conjunction with the accompanying drawings.

### BRIEF DESCRIPTION OF THE DRAWINGS

FIG. 1 shows a schematic of the synthesis of dual payload ADCs of the invention.

FIG. 2 demonstrates the cytotoxicity of dual-payload ADCs in comparison with mono-payload ADCs when incubated for 72 h with BXPC3 (Trop2+) cells.

FIG. 3 demonstrates the cytotoxicity of dual-payload ADCs in comparison with mono-payload ADCs when incubated for 72 h with BXPC3 (Trop2+) cells.

FIG. 4 shows the results of in vitro NFκB immune modulation studies of dual-payload ADCs of the invention in comparison with mono-loaded ADCs.

FIG. 5 shows the results of in vitro NFκB immune modulation studies of dual-payload ADCs of the invention in comparison with mono-loaded ADCs.

FIG. 6 shows the results of in vitro IRF immune modulation studies of dual-payload ADCs of the invention in comparison with mono-loaded ADCs.

FIG. 7 shows the results of in vitro IRF immune modulation studies of dual-payload ADCs of the invention in comparison with mono-loaded ADCs.

FIG. 8 shows the results of LCMS (top) and SEC (bottom) of a dual-payload ADC of the invention.

FIG. 9 shows the results of LCMS (top) and SEC (bottom) of a dual-payload ADC of the invention.

FIG. 10 shows the results of LCMS (top) and SEC (bottom) of a dual-payload ADC of the invention.

FIG. 11 shows the results of LCMS (top) and SEC (bottom) of a dual-payload ADC of the invention.

### DETAILED DESCRIPTION OF THE INVENTION

Chemistry has been developed to simultaneously attach both a TLR7 agonist (immune stimulant) and MMAF or MMAE to a single tumor-targeting antibody to form a multi-drug antibody-drug-conjugate (ADC). The ADC gets internalized into tumor tissue, releasing the drug, which permeates to nearby tissues, resulting in immunoactivation. With this co-delivery, the cytotoxic MMAF or MMAE is released upon internalization into the tumor cells, thereby killing the antigen-expressing cell and releasing neoantigens into the tumor microenvironment. Simultaneously, the immune stimulating payload (TLR7 agonist) is released into the tumor microenvironment where it increases the activity of tumor associated lymphocytes, macrophages and dendritic cells. The combination of these two mechanisms results in synergistic activity, thus enabling treatments of various tumors that are resistant to current therapeutic approaches. The dual mechanism of action imparts increased efficacy, decreased resistance, and/or increased spectrum of activity as compared to traditional mono-payload ADCs.

Several highly potent TLR agonists have been attached to antibody-directed tumor cells. Anti-tumor effects are driven by localized release of the TLR-agonist inducing an adaptive immune response against the tumor. Anti-pathogen effects are driven by attachment of the TLR agonist to an antibody that binds to the pathogen. The opsonized pathogen is then taken up by dendritic cells (antigen-presenting cells) where the TLR agonist is released—resulting in an enhanced adaptive immune response.

Changes in the conjugation method (i.e., permutation A and permutation B), the linker (i.e., cleavable or non-cleavable linker), and the payload (potency, permeability, etc.) effect changes in pharmacokinetics, efficacy and safety. Importantly, this disclosed technology does not require the introduction of non-natural amino acids or new cysteine residues into the antibody backbone, although non-natural amino acids or new cysteine residues can be utilized.

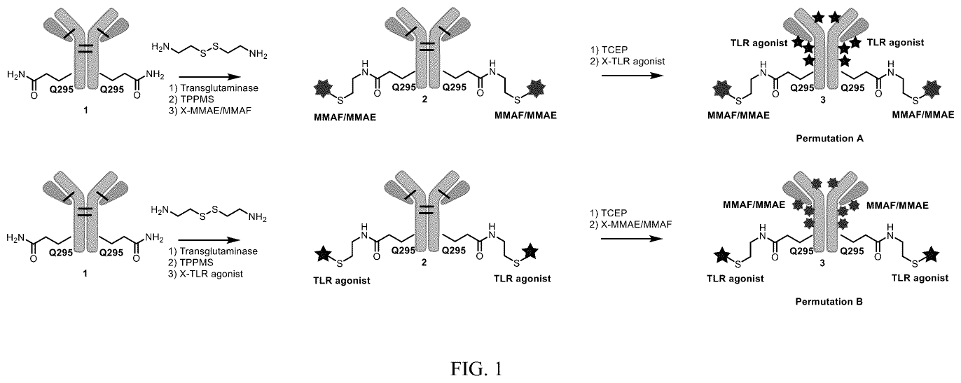

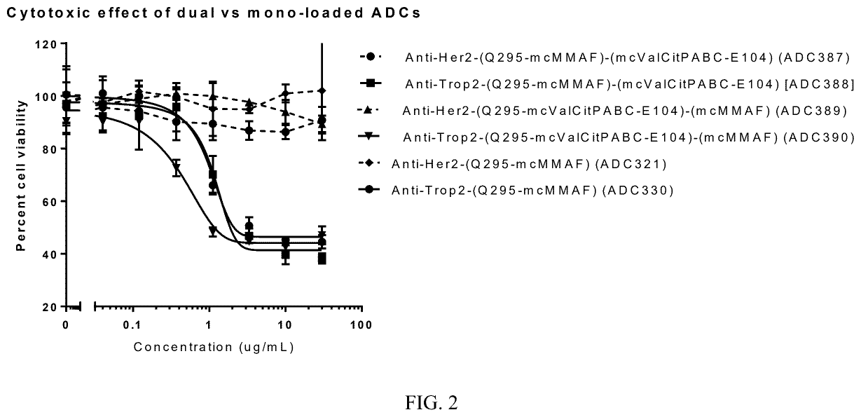

The chemistry, as shown in FIG. 2, utilizes the Q295 site for attachment of one of the payloads and the endogenous cysteine residues for the attachment of the other. This allows for a loading (drug-antibody ratio or “DAR”) of about 2 for one of the payloads and about 2 to 8 for the other payload. Attachment to the Q295 site is accomplished using a slight modification of published methodology (Benjamin S R et al. Mol Pharm. 2019 Jun. 3; 16(6):2795-2807. doi: 10.1021/acs.molpharmaceut.9b00323. Epub 2019 May 17. PMID: 31067063), as shown in FIG. 2. The antibody is first deglycosylated with PNGase F in order to expose the Q295 site, thereby making it amenable to transglutaminase-mediated conjugation of the thiol handle. Treatment of the intermediate with triphenylphosphine meta sulfonic acid (TPPMS) selectively reduces the disulfide at the Q295 site, thereby allowing conjugation of payload #1. Payload 1, represented herein by [P1-L^{P1}-J1-S^{Q}], is selectively loaded on the two Q295 sites of Ab, for example, as illustrated in FIG. 1. However, some additional extraneous loading at other heavy chain sites (i.e., non-Q295 sites) may result in a measured DAR value that is greater than the maximum claimed value for d1 of “about 2.” For instance, ADC630, described below, possesses a measured DAR value of 2.7, yet there are only a maximum of two Q295 sites on which the payload (mc_MMAF) may theoretically be loaded. Reduction with TCEP cleaves the intra-chain disulfides, thereby allowing conjugation of payload #2. The entire process can be expedited by capturing the deglycosylated antibody onto protein A resin, thus allowing the facile removal of excess reagents between each step. Both payloads are attached using cysteine-reactive groups (such as a maleimide or bromoacetamide), thus enabling two orthogonal designs, as shown in FIG. 2. In some examples of the present invention, payload #1 is MMAF or MMAE while payload #2 is a TLR agonist. Alternatively, payload #1 can be a TLR agonist while payload #2 is MMAF or MMAE:

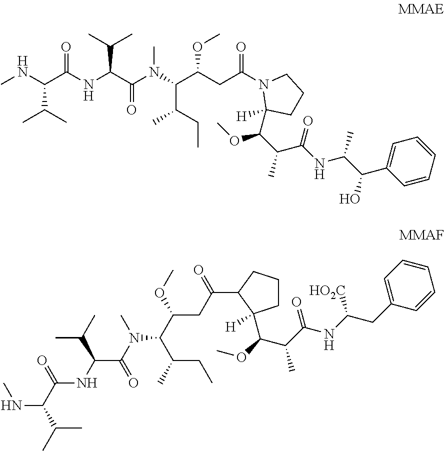 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C ; CC[C@H](C)[C@@H]([C@@H](CC(=O)C1CCC[C@H]1[C@H](OC)[C@@H](C)C(=O)N[C@@H](Cc1ccccc1)C(=O)O)OC)N(C)C(=O)[C@@H](NC(=O)[C@@H](NC)C(C)C)C(C)C]

In some embodiments, the antibody-drug conjugate is of Formula (A):

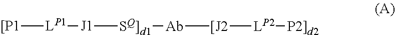

wherein:
P1 comprises a TLR7 agonist, MMAE, or MMAF;
L^{P1} comprises a cleavable or noncleavable linker;
J1 comprises a first conjugation handle;
S^{Q} is —S(C₁-C₆)alkylNH—, wherein the NH is attached to the carbonyl of a Q295 residue of Ab;
Ab comprises an antibody;
J2 comprises a second conjugation handle attached at a sulfur atom of a cysteine residue of Ab;
L^{P2} comprises a cleavable or noncleavable linker; and
P2 comprises a TLR7 agonist, MMAE or MMAF;
wherein:
d1 is between 1 and about 2;
d2 is between about 2 and about 10;
L^{P1} and L^{P2} may be the same or different;
J1 and J2 may be the same or different; and
when P1 is a TLR7 agonist, P2 is selected from MMAE or MMAF; and when P1 is MMAE or MMAF, P2 is a TLR7 agonist.

In some embodiments, d1 is between 1 and about 2. In some embodiments, d1 is 1. In some embodiments, d1 is about 2. In other embodiments, d1 is 2.

In some embodiments, d2 is between about 2 and about 10. In other embodiments, d2 is about 2, about 3, about 4, about 5, about 6, about 7, about 8, about 9, or about 10. In other embodiments, d2 is 2, 3, 4, 5, 6, 7, 8, 9, or 10. In other embodiments, d2 is between about 2 and about 8, about 2 and about 6, between about 2 and about 4, between about 4 and about 6, between about 4 and about 8, between about 4 and about 10, between about 6 and about 8, between about 6 and about 10, or between about 8 and about 10.

When d1 of the ADC is greater than 1, each P1 is the same moiety, as is each L^{P1}, each J1, and each S^{Q}. The same is true for each P2, each L^{P2}, and each J2. As a non-limiting example, if d2 is 4, then there will be four of the same drug, linker, and conjugation handle present attached to Ab. In rare occasions, d1 may be slightly greater than 2, due to minor non-specific attachment to residues other than Q295.

In some embodiments, P1 or P2 comprises MMAE. In some embodiments, P1 or P2 comprises MMAF. In other embodiments, P1 or P2 comprises a TLR7 agonist. In some embodiments, P1 comprises MMAE or MMAF and P2 comprises a TLR7 agonist. In other embodiments P1 comprises a TLR7 agonist and P2 comprises MMAE or MMAF.

In some embodiments, P1 or P2 is a TLR7 agonist of Formula (I) or Formula (II):

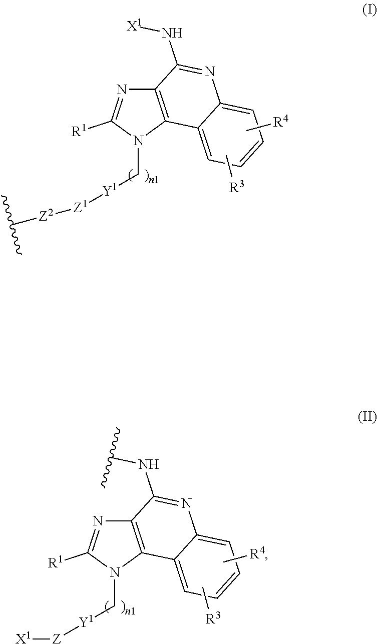

wherein:
R¹ is selected from C₁-C₁₀ alkyl, C₁-C₁₀ oxaalkyl, and C₁-C₁₀ azaalkyl;
R³ and R⁴ are each independently selected from hydrogen, C₁-C₅ alkyl, and C₁-C₅ alkoxy;
n1 is 1 or 2;
Y¹ is independently selected from optionally substituted aryl and optionally substituted heteroaryl;
Z¹ is selected from —NR^{Z}—, —O—, —NR^{Z}C(O)—, —NR^{Z}C(O)—O—, and —NR^{Z}SO₂—;
Z² is absent, or is selected from —(C₁-C₈)hydrocarbon, —(C₁-C₈)hydrocarbon-NH—, —NH—(C₁-C₈)hydrocarbon, and a 5-to 8-membered nitrogen-containing heterocycle, wherein the nitrogen is attached to L^{P1} or L^{P2}.
Z is independently selected from —NR^{Z}—, —NR^{Z}C(O)—, and —O—;
R^{Z} is independently selected in each instance from hydrogen, C₁-C₈ hydrocarbon, C₁-C₈ oxaalkyl, C₁-C₈ azaalkyl, heteroaryl, C₁-C₄ arylalkyl, and a 5-to 8-membered heterocyclic ring;
X¹ is independently selected from R^{Z}, —C(O)—R^{Z}, —C(O)—O—R^{Z}, —C(O)—N—(R^{Z})₂, —(CH₂)ₖNR^{Z}C(O)—(C₁-C₆)alkyl, —(CH₂)ₖNR^{Z}C(O)—O—(C₁-C₄)alkyl, —SO₂—NH—R^{Z}, and —SO₂—R^{Z}.
k is an integer from 1 to 8; and
[symbol] is the point of attachment to L^{P1} or L^{P2}.

In some embodiments, P1 or P2 is a TLR7 agonist of Formula (I):

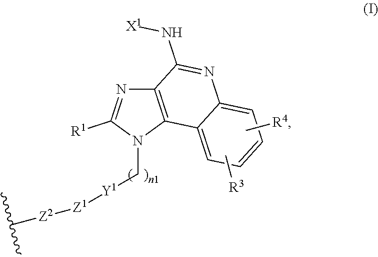

and [symbol] is the point of attachment to L^{P1} or L^{P2}.

In some embodiments, P1 or P2 is a TLR7 agonist of Formula (II):

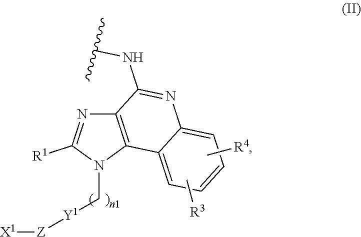

and [symbol] is the point of attachment to L^{P1} or L^{P2}.

In some embodiments, R¹ is C₁-C₁₀ alkyl. In other embodiments, R¹ is n-butyl. In some embodiments, R¹ is C₁-C₁₀ oxaalkyl. In other embodiments, R¹ is —CH₂OH. In still other embodiments, R¹ is —CH₂CH₂OH, or R¹ is —CH₂CH₂CH₂OH. In some embodiments, R¹ is —CH₂OCH₂CH₃. In other embodiments, R¹ is —CH₂OCH₂CH₂CH₃, or R¹ is —CH₂CH₂OCH₂CH₃, or R¹ is —CH₂CH₂OCH₃. In still other embodiments, R¹ is C₁-C₁₀ azaalkyl. In some embodiments, R¹ is —CH₂NHCH₂CH₃. In other embodiments, R¹ is —CH₂NHCH₂CH₂CH₃, or R¹ is —CH₂CH₂NHCH₂CH₃, or R¹ is-CH₂CH₂NHCH₃.

In some embodiments, R³ is hydrogen. In other embodiments, R³ is C₁-C₅ alkyl. In some embodiments, R³ is selected from methyl, ethyl, propyl, or butyl. In still other embodiments, R³ is C₁-C₅ alkoxy. In some embodiments, R³ is selected from methoxy, ethoxy, propoxy, or butoxy.

In some embodiments, R⁴ is hydrogen. In other embodiments, R⁴ is C₁-C₅ alkyl. In some embodiments, R⁴ is selected from methyl, ethyl, propyl, or butyl. In still other embodiments, R⁴ is C₁-C₅ alkoxy. In some embodiments, R⁴ is selected from methoxy, ethoxy, propoxy, or butoxy.

To be abundantly clear, R³ and R⁴ are independently selected. As non-limiting examples, both R³ and R⁴ may be hydrogen, both R³ and R⁴ may be a C₁-C₅ alkyl, or one of R³ and R⁴ may be C₁-C₅ alkoxy and the other of R³ and R⁴ may be hydrogen.

In some embodiments, n1 is 1. In some embodiments, n1 is 2.

In some embodiments, Y¹ is unsubstituted or substituted aryl. In other embodiments, Y¹ is unsubstituted or substituted phenyl. In some embodiments, Y¹ is unsubstituted or substituted heteroaryl. In other embodiments, Y¹ is unsubstituted or substituted pyridyl. In some embodiments, Y¹ is substituted with one or more of halogen, C₁-C₄ alkyl, C₁-C₄ alkoxy, C₁-C₄ haloalkyl, and/or C₁-C₄ haloalkoxy. In other embodiments, Y¹ is substituted with one or more of chloro, fluoro, methyl, ethyl, propyl, and/or methoxy.

In some embodiments, Z¹ is —NR^{Z}—. In other embodiments, Z¹ is —O—. In some embodiments, Z¹ is —NR^{Z}C(O)—. In some embodiments, Z¹ is —NR^{Z}C(O)—O—. In still other embodiments, Z¹ is —NR^{Z}SO₂—. In some embodiments, Z¹ is —NR^{Z}— or —O—.

In some embodiments, Z² is absent. In other embodiments, Z² is —(C₁-C₅)hydrocarbon-NH—. In still other embodiments, Z² is —(C₁-C₅)alkyl-NH—. In other embodiments, Z² is -benzyl-NH—. In yet other embodiments, Z² is -phenyl-NH—. In these embodiments wherein Z² is —(C₁-C₅)hydrocarbon-NH—, —(C₁-C₅)alkyl-NH—, or -benzyl-NH—, the nitrogen is attached to L^{P1} or L^{P2}. In some embodiments, Z² is a 5-to 8-membered nitrogen-containing heterocycle, wherein a nitrogen of the heterocycle is attached to L^{P1} or L^{P2}. In some embodiments, Z² is —(C₁-C₈)hydrocarbon. In other embodiments, Z² is benzyl. In still other embodiments, Z² is —(C₁-C₈)alkyl. In some embodiments, Z² is —NH—(C₁-C₅)hydrocarbon-. In still other embodiments, Z² is —NH—(C₁-C₈)alkyl-. In other embodiments, Z² is —NH-benzyl-. In yet other embodiments, Z² is —NH-phenyl-.

In some embodiments, Z is —O—. In other embodiments, Z is —NR^{Z}—. In still other embodiments, Z is —NR^{Z}C(O)—.

In some embodiments, X¹ is R^{Z}. In some embodiments, X¹ is hydrogen. In some embodiments, X¹ is methyl. In other embodiments, X¹ is C(O)—R^{Z}. In still other embodiments, X¹ is C(O)—O—R^{Z}. In some embodiments, X¹ is C(O)—N—(R^{Z})₂. In other embodiments, X¹ is SO₂—R^{Z}. In other embodiments, X¹ is —(CH₂)ₖNR^{Z}C(O)—(C₁-C₆)alkyl. In yet other embodiments, X¹ is —(CH₂)ₖNR^{Z}C(O)—O—(C₁-C₄)alkyl. In still other embodiments, X¹ is —SO₂—NH—R^{Z}.

In some embodiments, R^{Z} is hydrogen. In other embodiments, R^{Z} is C₁-C₈ hydrocarbon. In still other embodiments, R^{Z} is C₁-C₈ alkyl. In some embodiments, R^{Z} is methyl. In other embodiments, R^{Z} is C₁-C₈ oxaalkyl. In still other embodiments, R^{Z} is C₁-C₈ azaalkyl. In yet other embodiments, R^{Z} is —C(NH₂)benzyl. In some embodiments, R^{Z} is heteroaryl. In some embodiments, R^{Z} is a 5-to 8-membered heterocyclic ring. In other embodiments, R^{Z} is C₁-C₄ arylalkyl. In some of these embodiments, R^{Z} is C₁-phenyl, C₂-phenyl, C₃-phenyl, or C₄-phenyl. Each instance of R^{Z} is independently selected. As non-limiting illustrative examples, if Z is —NR^{Z}— and X¹ is —C(O)—R^{Z}, both instances of R^{Z} may be hydrogen, or Z may be —NCH₂— and X¹ may be —C(O)CH₃, or Z may be —NH— and X¹ may be —C(O)CH₂CH₃.

In some embodiments, k is an integer from 1 to 8. In some embodiments, k is an integer from 1 to 6. In some embodiments, k is an integer from 1 to 4. In some embodiments, k is an integer from 1 to 3. In some embodiments, k is an integer from 1 to 2. In some embodiments, k is an integer from 2 to 4. In some embodiments, k is an integer from 2 to 3. In some embodiments, k is 1. In some embodiments, k is 2. In some embodiments, k is 3. In some embodiments, k is 4. In some embodiments, k is 5. In some embodiments, k is 6. In some embodiments, k is 7. In some embodiments, k is 8.

In some embodiments, P1 or P2 is a TLR7 agonist of one of the structures shown below:

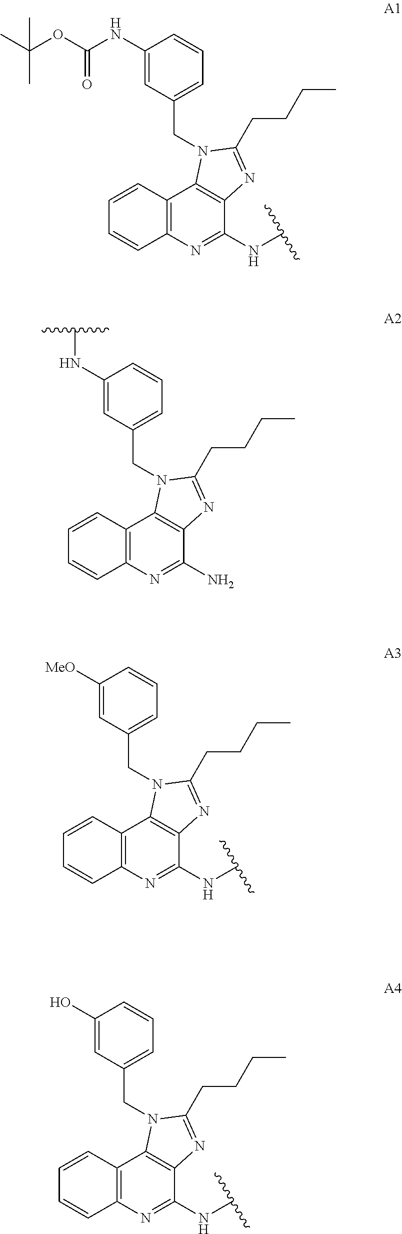

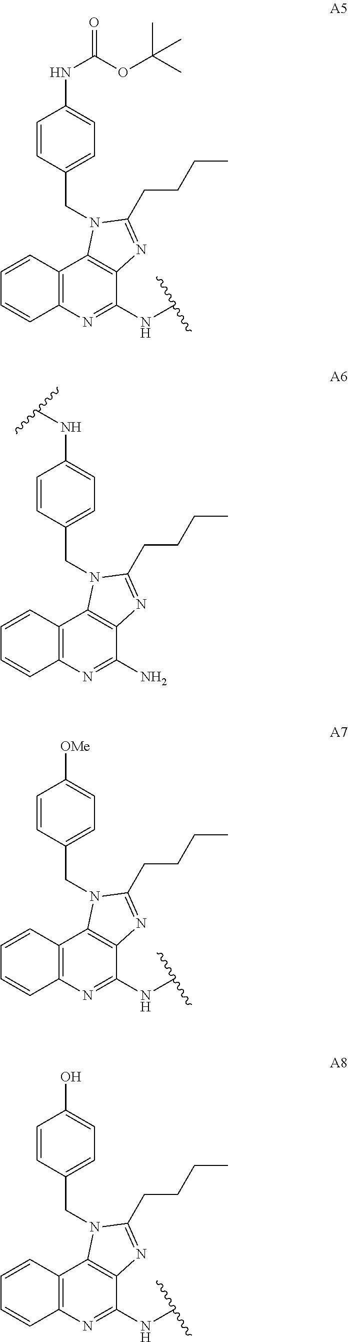

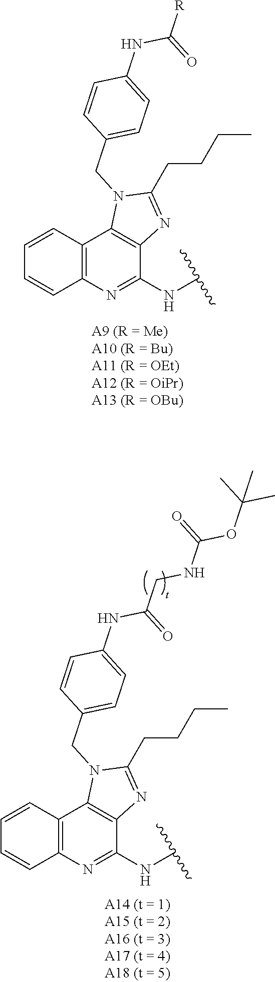

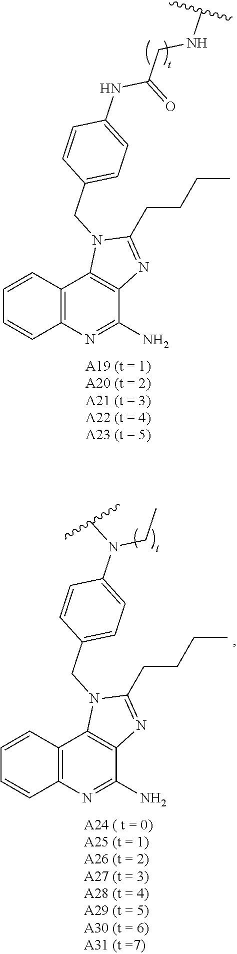

wherein [symbol] is the point of attachment to L^{P1} or L^{P2}. In other embodiments, P1 or P2 is a TLR7 agonist of one of structures A6, A10, A12, A13, or A15 shown above. In still other embodiments, P1 or P2 is a TLR7 agonist of one of structures A6, A10, or A15 shown above.

In some embodiments when the TLR7 agonist is of Formula (I), X¹ is hydrogen, n1 is 1, and Y¹ is phenyl, the TLR7 agonist is of formulae (Ia), (Ib), (Ic), or (Id):

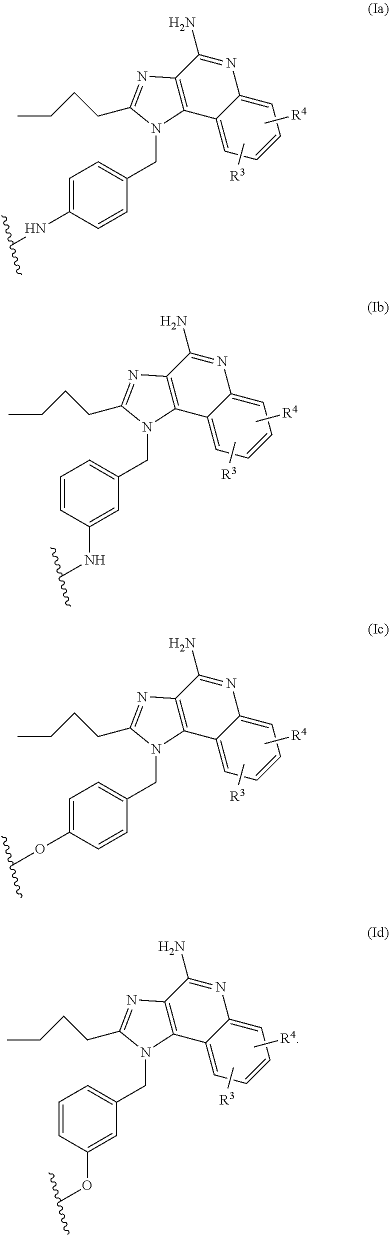

In these embodiments of formulae (Ia), (Ib), (Ic), or (Id) [symbol] represents a point of attachment to L^{P1} or L^{P2}.

In some embodiments when the TLR7 agonist is of Formula (II), n1 is 1, and Y¹ is phenyl, the TLR7 agonist is of formulae (Iia), (Iib), (Iic), or (Iid):

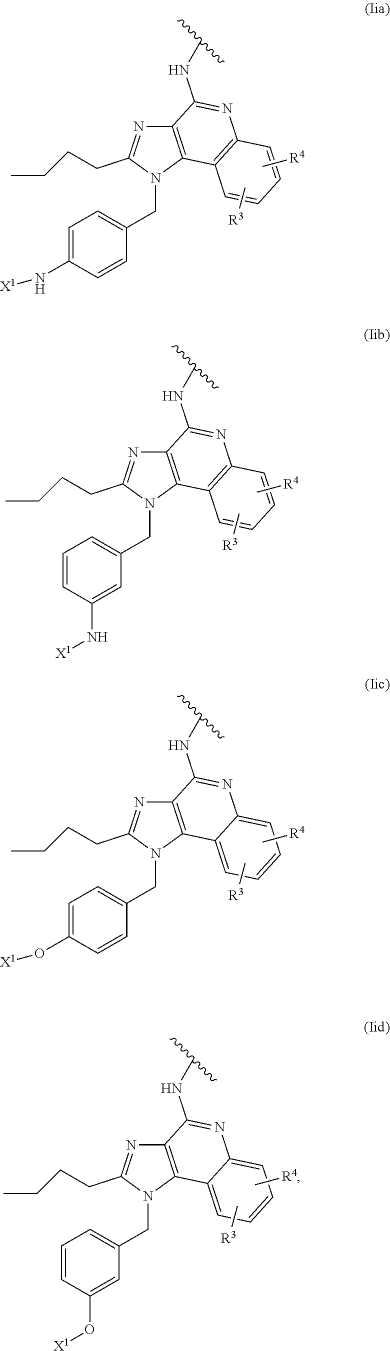

wherein [symbol] represents a point of attachment to L^{P1} or L^{P2}.

In some embodiments, P1 or P2 is of Formula (I), and:
R¹ is n-butyl;
R³ and R⁴ are each hydrogen;
n1 is 1;
Y¹ is phenyl or pyridyl, each of which is unsubstituted or substituted with one or more of halogen, C₁-C₄ alkyl, C₁-C₄ alkoxy, C₁-C₄ haloalkyl, or C₁-C₄ haloalkoxy;
X¹ is hydrogen; and
Z¹ is —N(R^{Z})— or —O—.

In some embodiments, P1 or P2 is one of the structures shown below:

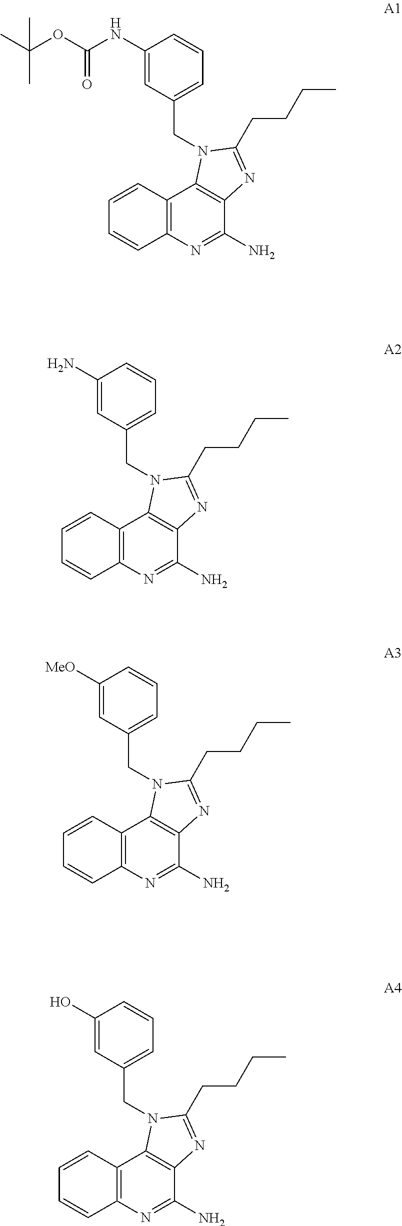 [SMILES: CCCCc1nc2c(N)nc3ccccc3c2n1Cc1cccc(NC(=O)OC(C)(C)C)c1 ; CCCCc1nc2c(N)nc3ccccc3c2n1Cc1cccc(N)c1 ; CCCCc1nc2c(N)nc3ccccc3c2n1Cc1cccc(OC)c1 ; CCCCc1nc2c(N)nc3ccccc3c2n1Cc1cccc(O)c1]

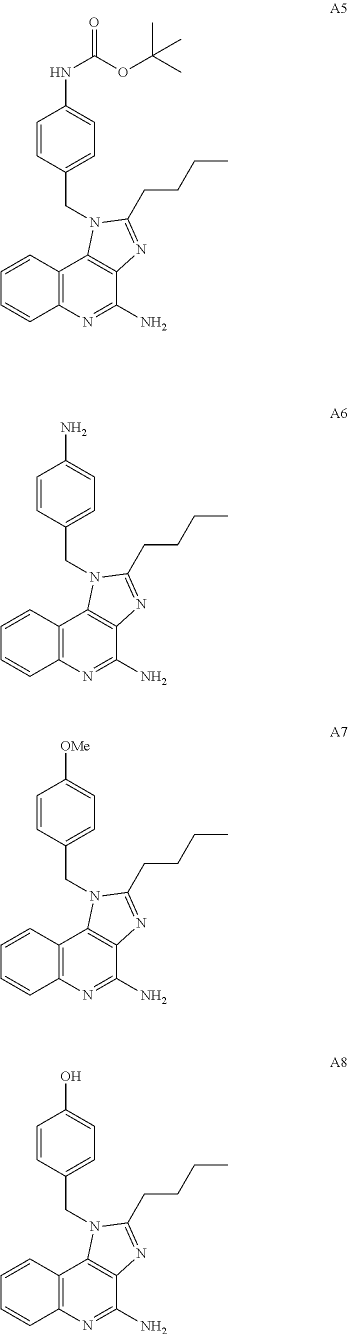 [SMILES: CCCCc1nc2c(N)nc3ccccc3c2n1Cc1ccc(NC(=O)OC(C)(C)C)cc1 ; CCCCc1nc2c(N)nc3ccccc3c2n1Cc1ccc(N)cc1 ; CCCCc1nc2c(N)nc3ccccc3c2n1Cc1ccc(OC)cc1 ; CCCCc1nc2c(N)nc3ccccc3c2n1Cc1ccc(O)cc1]

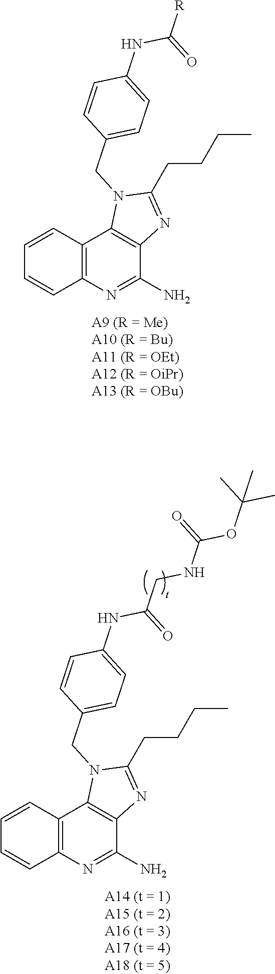

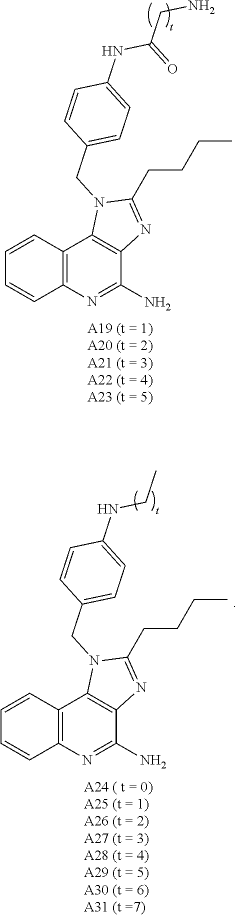

In other embodiments, P1 or P2 is a TLR7 agonist of one of structures A6, A10, A12, A13, or A15 shown above. In still other embodiments, P1 or P2 is a TLR7 agonist of one of structures A6, A10, or A15 shown above.

In some embodiments, S^{Q} is —S(C₂-C₆)alkylNH—, wherein the NH is attached to the carbonyl of a Q295 residue of Ab. The (C₂-C₆)alkyl moiety of S^{Q} may be a straight chain or branched. In some embodiments, the (C₂-C₆)alkyl moiety of S^{Q} is ethyl, propyl, isopropyl, n-butyl, s-butyl, or t-butyl. In some embodiments, the (C₂-C₆)alkyl moiety of S^{Q} is —CH₂CH₂—, —CH(CH₃)CH₂—, —CH₂CH₂CH₂—, —CH₂CH(CH₃)—, —CH(CH₃)CH₂CH₂—, —CH(CH₃)CH(CH₃)—, —CH₂CH(CH₃)CH₂—, or —CH₂CH₂CH(CH₃)—. In other embodiments, the (C₁-C₆)alkyl moiety of S^{Q} is ethyl.

In some embodiments, n1 is 1, Y¹ is optionally substituted aryl, Z¹ is —NR^{Z}—, and Z² is absent. In some of these embodiments, R^{Z} is hydrogen. In other embodiments, n2 is 1, Y¹ is optionally substituted heteroaryl, Z¹ is —NR^{Z}—, and Z² is absent. In some of these embodiments, R^{Z} is hydrogen. In some embodiments, n1 is 2, Y¹ is optionally substituted aryl, Z¹ is —NR^{Z}—, and Z² is absent. In some of these embodiments, R^{Z} is hydrogen. In some embodiments, n1 is 2, Y¹ is optionally substituted heteroaryl, Z¹ is —NR^{Z}—, and Z² is absent. In some of these embodiments, R^{Z} is hydrogen. In some of these embodiments, Y¹ is optionally substituted phenyl. In some of these embodiments, Y¹ is unsubstituted phenyl.

In this disclosure, amino acid residue numbering is based on the EU numbering system (Edelman et al. (1969) Biochemistry Vol. 63, pp 78-85). The “Q295” residue is glutamine 295 as defined as the Q in the portion of the IgG peptide sequence “KPREEQ(Y/F)NSTYR.” This could also include sequences that have about 90% or greater homology. The percent homology indicates an amino acid similarity determined using one of the BLAST (Basic Local Alignment Search Tool) family of programs (see, e.g., Altschul et al., 1990, J. Mol. Biol. 215, 403-410 or Altschul et al. (1997), Nucleic Acids Res, 25:3389-3402).

In an ADC the linker serves to attach the payload to the antibody.

The linker may be cleavable, consisting of a chemically labile linker including acid-cleavable linkers and reducible linkers or an enzyme cleavable linker such as peptide-based linkers or glucuronide linkers well known in the art. In one embodiment, the linker is cleavable via intracellular enzymes (e.g., cathepsin-B or Legumain).

In one embodiment, a second section of the linker unit is introduced which has a second reactive site e.g., an electrophilic group that is reactive to a nucleophilic group present on an antibody unit (e.g., an antibody). Useful nucleophilic groups on an antibody include but are not limited to, sulfhydryl, hydroxyl, and amino groups. The heteroatom of the nucleophilic group of an antibody may be reactive to an electrophilic group on a linker unit and forms a covalent bond to a linker unit. Useful electrophilic groups include, but are not limited to, maleimide, haloacetamide, and activated ester groups. The electrophilic group may provide a convenient site for antibody attachment.

In another embodiment, a linker unit has a reactive site which has a nucleophilic group that is reactive to an electrophilic group present on an antibody. Useful electrophilic groups on an antibody include, but are not limited to, aldehyde and ketone carbonyl groups. The heteroatom of a nucleophilic group of a linker unit can react with an electrophilic group on an antibody and form a covalent bond to the antibody. Useful nucleophilic groups on a linker unit include, but are not limited to, hydrazide, oxime, amino, hydrazine, thiosemicarbazone, hydrazine carboxylate, and arylhydrazide. The electrophilic group on an antibody may provide a convenient site for attachment to a linker unit. In another embodiment, a linker unit has a functionality that can be attached to the antibody through an enzymatic reaction. One particularly useful example of this is the transamidation of amine-containing linkers with glutamine, a reaction that is promoted by bacterial transglutaminase. This reaction can be used to attach payloads to endogenous glutamine residues, as in Benjamin et al. (Mol. Pharmaceutics 2019, 16, 6, 2795-2807) or may be used to attach payloads to specifically engineered glutamine tags, as in Strop et al. (Chemistry and Biology 2013, 20, 2, 161-167), both of which are hereby incorporated by reference in their entirety.

Amino functional groups are also useful reactive sites for a linker unit because they can react with carboxylic acid, or activated esters of a compound to form an amide linkage. The peptide-based compounds of the present disclosure may, in one embodiment, be prepared by forming a peptide bond between two or more amino acids and/or peptide fragments. Such peptide bonds can be prepared, for example, according to the liquid phase synthesis method (see, e.g., Schroder and Lubke, The Peptides, 1^{st} Ed., pp 76-136 (Academic Press 1966), which is hereby incorporated by reference in its entirety) that is well known in the field of peptide chemistry.

In the context of the present disclosure, particularly but not limited to linker components, the language “selected from one or more of” or “one or more of” indicates that multiple components, which may be the same or different, are or may be arranged sequentially. Thus, for example, L^{P1} or L^{P2} may be any individually or combined listed components.

In one embodiment, L^{P1} and L^{P2} of the antibody-drug conjugate of Formula (A) is a cleavable or noncleavable linker (referred to herein as “linker” or “L^{P1}” or “L^{P2}” as further described herein). In some embodiments, L^{P1} comprises a cleavable or noncleavable linker. In some embodiments, L^{P2} comprises a cleavable or noncleavable linker.

In some embodiments, L^{P1} and L^{P2} are each independently selected from:

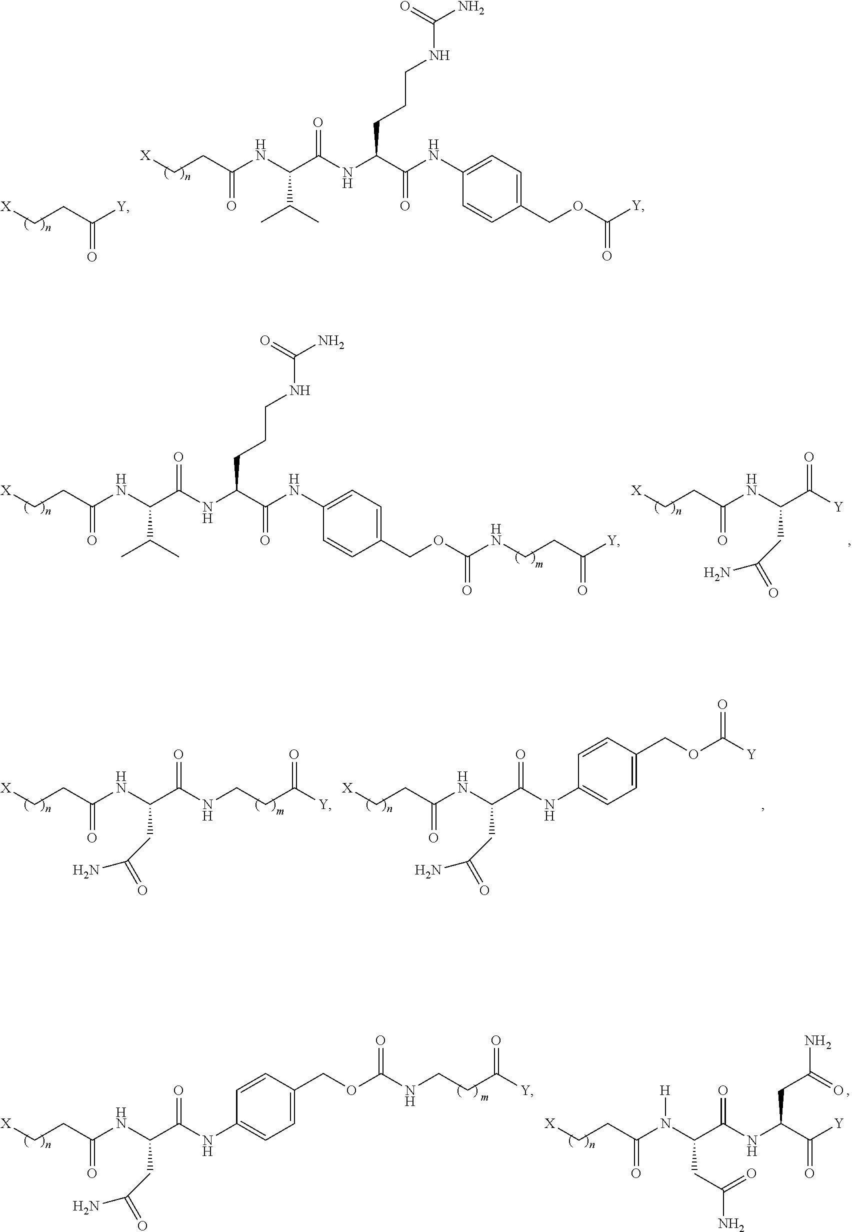

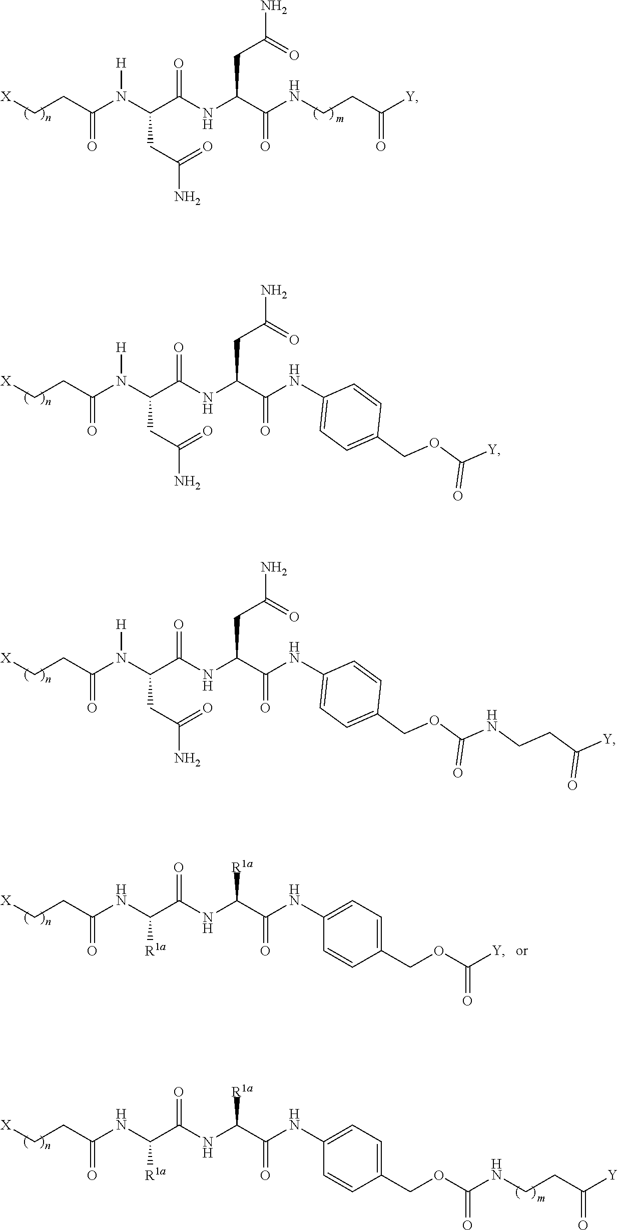

wherein:
R^{1a} is hydrogen, (C₁-C₄)alkyl, benzyl, CH₂OH, (CH₂)ᵥNH₂, CH₂CO₂H, CH₂CH₂CO₂H, CH₂CONH₂, CH₂CH₂CONH₂;
v is 2, 3, 4, or 5;
m is 0, 1, 2, 3, 4, 5, 6, or 7;
n is 0, 1, 2, 3, 4, 5, 6, or 7;
Y is P1 or P2; and
X is J1 or J2.

To be clear, L^{P1} and L^{P2} may be the same as each other or may be different from each other. For instance, in one non-limiting example of an ADC of Formula (A), L^{P1} is

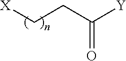

and n is 2, while L^{P2} is

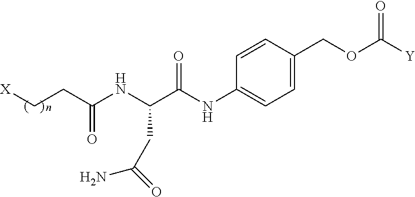

and n is 1. However, as explained above, every L^{P1} in a single ADC of Formula (A) is the same, and every L^{P2} in a single ADC of Formula (A) is the same, no matter the values of d1 or d2.

In one embodiment, J1 and J2 are each independently selected from:

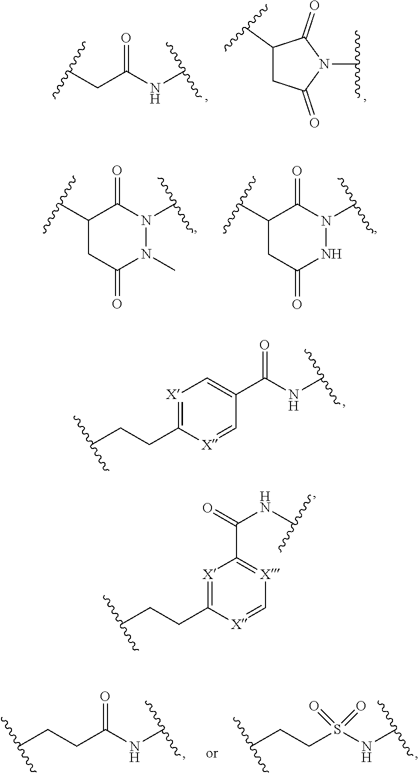

In these embodiments, X′, X″, and X′″ are each independently selected in each instance from CH or N; and the left-hand [symbol] is the point of attachment to Ab, and the right-hand [symbol] is the point of attachment to L^{P1} or L^{P2}.

To be clear, J1 and J2 may be the same as each other or may be different from each other. For instance, in one non-limiting example of an ADC of Formula (A), J1 is

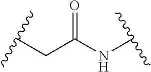 [SMILES: *CC(=O)N*]

### and J2 is

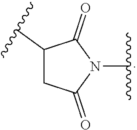 [SMILES: *C1CC(=O)N(*)C1=O]

However, as explained above, every J1 in a single ADC of Formula (A) is the same, and every J2 in a single ADC of Formula (A) is the same, no matter the values of d1 or d2.

In accordance with the present disclosure, the linker is defined as L^{P1} or L^{P2} and, in some embodiments, L^{P1}-J1 or J2-L^{P2} is L1-L2-(L3)ₚ-(L4)_{q}-(L5)ᵣ. In some embodiments, L^{P1}-J1 or J2-L^{P2} are each independently selected from L1-L2-(L3)ₚ-(L4)_{q}-(L5)ᵣ.

In accordance with one embodiment of the present disclosure, L1 is a conjugation moiety. A conjugation moiety as described herein includes a moiety that attaches L2 as described herein to a cysteine, lysine, or glutamine residue. In some embodiments, the glutamine is glutamine 295. Examples of L1 include maleimide, bromoacetamide, amine, NHS-ester, and the like.

In one embodiment, L2 as described herein is a spacer unit selected from branched or unbranched C₁-C₁₂ alkyl, a PEG selected from PEG1 to PEG12,

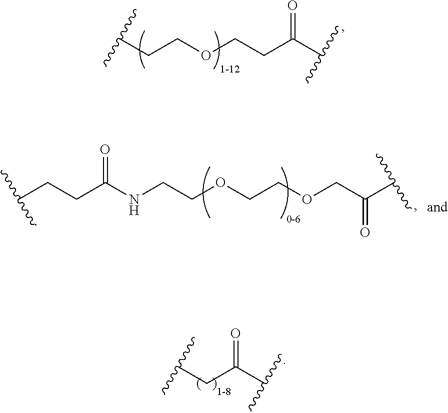

PEG may, for example, be PEG1, PEG2, PEG3, PEG4, PEG5, PEG6, PEG7, PEG8, PEG9, PEG10, PEG11, PEG12, or any combination thereof.

In one embodiment, L3 as described herein relates to a peptide of 1 to 6 amino acids. For example, the peptide may be 1 amino acid, 2 amino acids, 3 amino acids, 4 amino acids, 5 amino acids, or 6 amino acids. Amino acids may be selected both from natural amino acids and non-natural α-amino acids.

In one embodiment, L4 is a self-immolative spacer.

In one embodiment, L5 is carbonyl, as described herein.

In one embodiment, p, q, and r of the compound of Formula (I) and Formula (II) are each independently selected from 0 and 1.

In one embodiment, the compound of Formula (I) or Formula (II) may include L1 selected from:

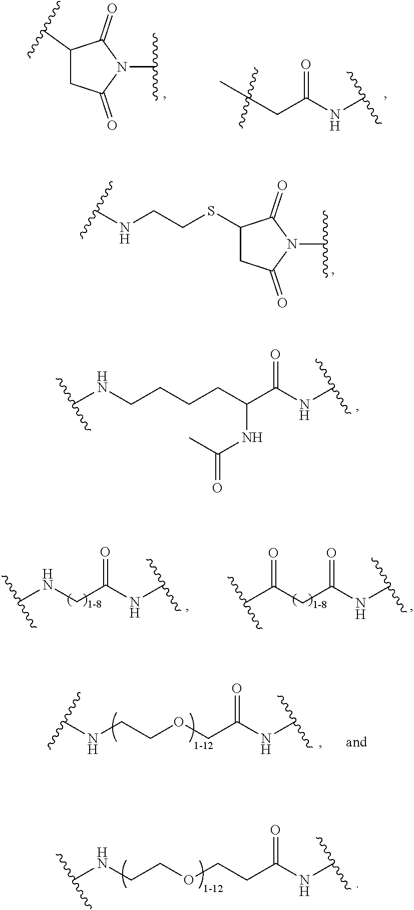

In another embodiment, the compound of Formula (I) or Formula (II) may include L2 selected from

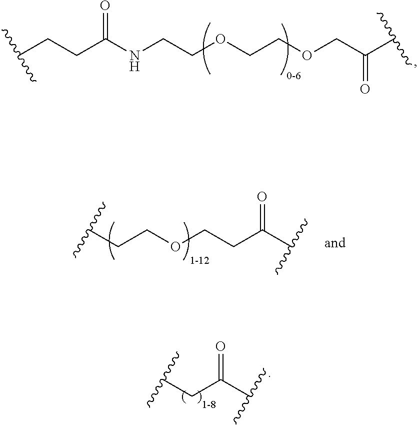

In yet another embodiment, the compound of Formula (I) or Formula (II) may include L3 of

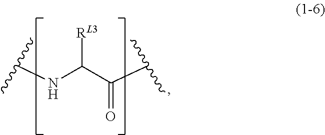

wherein R^{L3} is an amino acid side chain. In yet another embodiment, the compound of Formula (I) or Formula (II) may include L4 selected from:

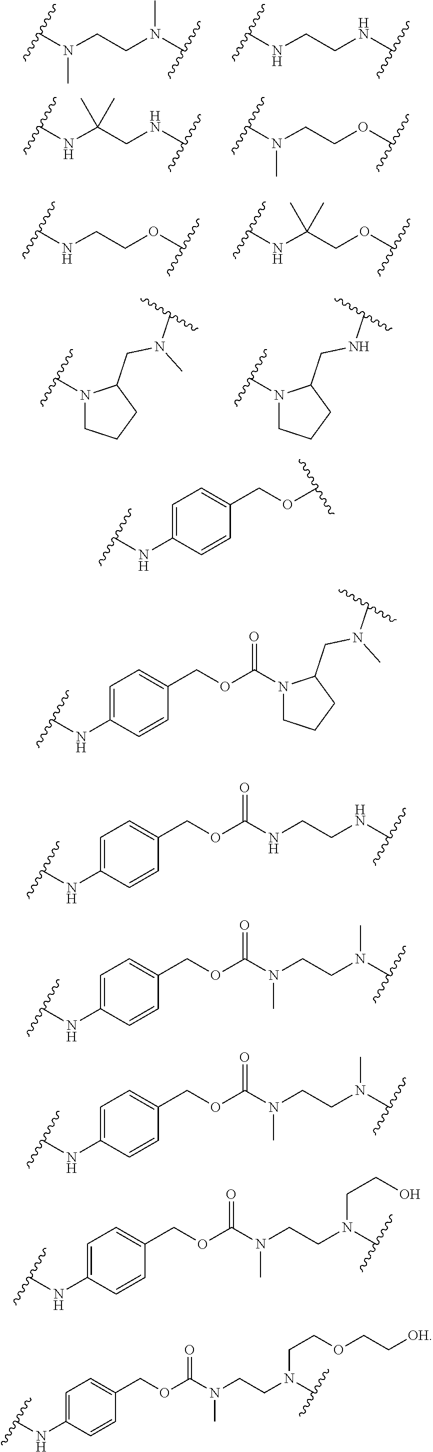

In yet another embodiment, the compound of Formula (I) or Formula (II) may include L5 of

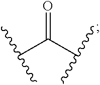 [SMILES: *C(*)=O]

and p, q, and r are each independently 0 or 1, wherein when q is 0 then r is 0, or when p is 1 then r is 1.

L3 may, in certain embodiments, include, but is not limited to, ValCit, GlyValCit, ValArg, PheLys, AlaAla, GlyGlyPheGly, AlaAlaAla, AlaAsn, AsnAsn, AsnAla, ValCitGlyPro, AsnGlyPro, AsnAsnGlyPro, Asn, GlyAsn, AsnAla, ProCitAla, ProAsnLeu, ProAsnAla, ProPheAla, ProPheGly, ProCitLeu, ProAsnPro, ProAsnSer, and ProAsnGly.

In one embodiment, the compound of Formula (I) or Formula (II) may include L1 that is selected from

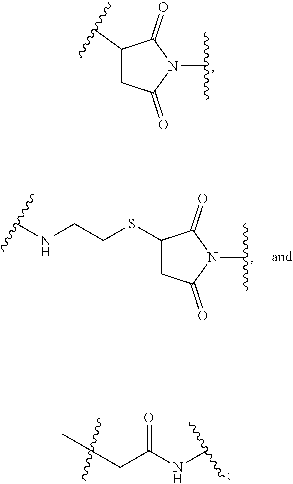 [SMILES: *C1CC(=O)N(*)C1=O ; *NCCSC1CC(=O)N(*)C1=O ; *CC(=O)N*]

L2 that is

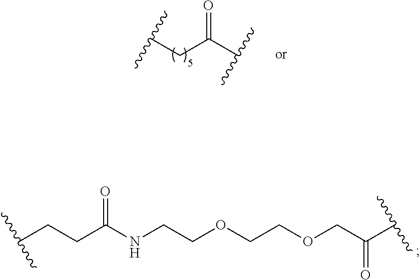

L3 that is ValCit, GlyValCit, AsnAsn, Asn or AlaAla; L4 that is selected from:

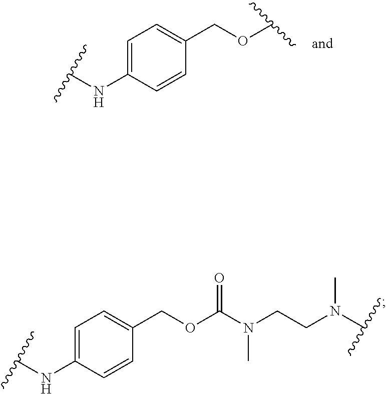 [SMILES: *Nc1ccc(CO*)cc1 ; *Nc1ccc(COC(=O)N(C)CCN(*)C)cc1]

and/or L5 that is

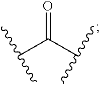 [SMILES: *C(*)=O]

and p, q, and r are each 0 or p, q, and r are each 1.

In some embodiments, L^{P1}-J1 or J2-L^{P2} is selected from mc, mcValCitPABC, mcAsnAsnGlyPro, mcValCitGlyPro, mcAlaAlaGlyPro, mcAsnAsnPABC, mcAsnAsn, mpAsnAsn, maAsnAsn, or mcAlaAlaPABC. In some embodiments, L^{P1}-J1 or J2-L^{P2} is selected from mcValCitPABC, mcAsnAsn, or mc. In some embodiments, L^{P1}-J1 or J2-L^{P2} is mcValCitPABC. In some embodiments, L^{P1}-J1 or J2-L^{P2} is mc. In some embodiments, L^{P1}-J1 or J2-L^{P2} is mcAsnAsn. In some embodiments, L^{P1}-J1 or J2-L^{P2} is mcValCitPABC and the other of L^{P1}-J1 or J2-L^{P2} is mc.

In some embodiments, L^{P1}-J1 or J2-L^{P2} is selected from mc, mcValCitPABC, mcAsnAsnGlyPro, mcValCitGlyPro, mcAlaAlaGlyPro, mcAsnAsnPABC, mcAsnAsn, mpAsnAsn, maAsnAsn, or mcAlaAlaPABC; and one of P1 or P2 is selected from a TLR agonist selected from structures A1 to A31 described above, while the other of P1 or P2 is selected from MMAE or MMAF. In some embodiments, L^{P1}-J1 or J2-L^{P2} is selected from mc, mcValCitPABC, mcAsnAsnGlyPro, mcValCitGlyPro, mcAlaAlaGlyPro, mcAsnAsnPABC, mcAsnAsn, or mcAlaAlaPABC; and one of P1 or P2 is selected from a TLR agonist selected from structures A6, A10, or A15 described above, while the other of P1 or P2 is selected from MMAE or MMAF. In some embodiments, P1 is the TLR agonist and P2 is MMAE or MMAF. In other embodiments, P2 is the TLR agonist and P1 is MMAE or MMAF.

In some embodiments, L^{P1}-J1 or J2-L^{P2} is selected from mcValCitPABC, mcAsnAsn, mpAsnAsn, maAsnAsn, or mc, and one of P1 or P2 is selected from a TLR agonist selected from structures A1 to A31 described above, while the other of P1 or P2 is selected from MMAE or MMAF. In some embodiments, L^{P1}-J1 or J2-L^{P2} is selected from mcValCitPABC, mcAsnAsn, or mc, and one of P1 or P2 is selected from a TLR agonist selected from structures A6, A10, or A15 described above, while the other of P1 or P2 is selected from MMAE or MMAF. In some embodiments, P1 is the TLR agonist and P2 is MMAE or MMAF. In other embodiments, P2 is the TLR agonist and P1 is MMAE or MMAF.

Additional, non-limiting combinations of P1-L^{P1}-J1 or J2-L^{P2}-P2 include mc-A6, me-A10, me-A15, me-MMAF, me-MMAE, mcValCitPABC-A6, mcValCitPABC-A10, mcValCitPABC-A15, mcAsnAsn-A6, mcAsnAsn-A10, mcAsnAsn-A15, mcValCitPABC-MMAF, mcValCitPABC-MMAE, mcAsnAsnGlyPro-A6, mcAsnAsnGlyPro-A10, mcAsnAsnGlyPro-A15, mcAsnAsnGlyPro-MMAF, mcAsnAsnGlyPro-MMAE, mcValCitGlyPro-A6, mcValCitGlyPro-A10, mcValCitGlyPro-A15, mpAsnAsn-A6, mpAsnAsn-A10, mpAsnAsn-A15, maAsnAsn-A6, maAsnAsn-A10, maAsnAsn-A15, mcValCitGlyPro-MMAF, mcValCitGlyPro-MMAE, mcAlaAlaGlyPro-A6, mcAlaAlaGlyPro-A10, mcAlaAlaGlyPro-A15, mcAlaAlaGlyPro-MMAF, mcAlaAlaGlyPro-MMAE, mcAsnAsnPABC-A6, mcAsnAsnPABC-A10, mcAsnAsnPABC-A15, mcAsnAsnPABC-MMAF, mcAsnAsnPABC-MMAE, mcAlaAlaPABC-A6, mcAlaAlaPABC-A10, mcAlaAlaPABC-A15, mcAlaAlaPABC-MMAF, or mcAlaAlaPABC-MMAE.

In some embodiments, L^{P1}-J1 is mcValCitPABC. In some of these embodiments, P1 is structure A6, structure A10, structure A15, MMAF, or MMAE. In some embodiments, L^{P1}-J1 is mcAsnAsn. In some of these embodiments, P1 is structure A6, structure A10, structure A15, MMAF, or MMAE. In other embodiments, L^{P1}-J1 is mc. In some of these embodiments, P1 is structure A6, structure A10, structure A15, MMAF, or MMAE. In some embodiments, L^{P2}-J2 is mcValCitPABC. In some of these embodiments, P2 is structure A6, structure A10, structure A15, MMAF, or MMAE. In some embodiments, L^{P2}-J2 is mcAsnAsn. In some of these embodiments, P2 is structure A6, structure A10, structure A15, MMAF, or MMAE. In other embodiments, L^{P2}-J2 is mc. In some of these embodiments, P2 is structure A6, structure A10, structure A15, MMAF, or MMAE.

In some embodiments, P1-L^{P1}-J1 is mcValCitPABC-A6. In some embodiments, P1-L^{P1}-J1 is mcAsnAsn-A6. In other embodiments, P1-L^{P1}-J1 is me-A6. In some embodiments, P1-L^{P1}-J1 is me-MMAF. In other embodiments, P1-L^{P1}-J1 is me-MMAE. In some embodiments, J2-L^{P2}-P2 is mcValCitPABC-A6. In some embodiments, J2-L^{P2}-P2 is mcAsnAsn-A6. In other embodiments, J2-L^{P2}-P2 is me-A6. In some embodiments, J2-L^{P2}-P2 is me-MMAF. In other embodiments, J2-L^{P2}-P2 is me-MMAE.

Unless explicitly specified, compounds of Formula (I) or Formula (II) include compounds of formulae (Ia), (Ib), (Ic), (Id), (Iia), (Iib), (Iic), or (Iid). Unless explicitly specified, the embodiments described herein relate to any of Formula (I) or Formula (II), or formulae (Ia), (Ib), (Ic), (Id) (Iia), (Iib), (Iic), or (Iid).

In one aspect, the present invention provides a pharmaceutical composition comprising a compound described herein and a pharmaceutically acceptable carrier, diluent, or excipient. In one embodiment, the pharmaceutical composition further comprises a therapeutically effective amount of a chemotherapeutic agent.

In one aspect, the present invention provides a method for stimulating an immune response in a subject. The method includes administering a therapeutically effective amount of a compound described herein under conditions effective to stimulate an immune response. In some embodiments, the method is performed on a subject having cancer. In other embodiments, the cancer is bladder cancer, breast cancer, cervical cancer, colon cancer, endometrial cancer, kidney cancer, lung cancer, esophageal cancer, ovarian cancer, prostate cancer, pancreatic cancer, skin cancer, gastric cancer, testicular cancer, biliary cancer, colorectal cancer, endometrial cancer, head/neck cancer, medullary thyroid cancer, renal cancer, eye cancer, neuroblastoma, Mycosis fungoides, glial tumor, other brain tumor, spinal cord tumor, liver cancer, leukemia, lymphoma, or any combination thereof.

In certain embodiments, the immunotherapy compounds present in a liquid pharmaceutical composition are administered into a tumor (e.g., intratumoral (IT) administration) and induce an innate immune response and a cell-mediated immune response against the tumor antigens (e.g., shrink or stabilize the tumor). The conjugate comprising a peptide is not necessarily an antigen or immunogen, but a mechanism to reduce the solubility of the TLR7 and/or TLR8 agonist creating a depot that is retained at the site of administration, such as within a tumor or in the tumor microenvironment. The conjugated TLR7 and/or TLR8 agonist may stimulate immunosuppressive cells and may induce the immune response against the antigens present in the tumor. Moreover, mobilization of the immunosuppressive cells may induce an immune response against not only the tumor at the site of administration, but peripheral, nearby and/or distant tumors as well. In one embodiment, methods of stimulating an anti-tumor immune response in a subject are disclosed, where the methods comprise locally administering intratumorally or peritumorally a liquid form of the pharmaceutical composition into the subject, where the anti-tumor immune response is effective at a distant site from the site of administration of the pharmaceutical composition.

The present invention provides, in a fourth aspect, a method for inducing an anti-tumor immune response in a subject. The method includes administering a therapeutically effective amount of a compound described herein under conditions effective to induce an anti-tumor immune response. In some embodiments, the method is performed on a selected subject having a tumor. In some embodiments, the tumor is fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, or retinoblastoma.

The present invention provides, in a fifth aspect, a method for treating a tumor or abnormal cell proliferation in a subject. The method includes administering a therapeutically effective amount of a compound described herein under conditions effective to treat a tumor or abnormal cell proliferation. In some embodiments, the tumor or abnormal cell proliferation is cancer. In some embodiments, the cancer is bladder cancer, breast cancer, cervical cancer, colon cancer, endometrial cancer, kidney cancer, lung cancer, esophageal cancer, ovarian cancer, prostate cancer, pancreatic cancer, skin cancer, gastric cancer, testicular cancer, biliary cancer, colorectal cancer, endometrial cancer, head and neck cancer, medullary thyroid cancer, renal cancer, eye cancer, neuroblastoma, Mycosis fungoides, glial tumor, other brain tumor, spinal cord tumor, liver cancer, leukemia, lymphoma, or any combination thereof.

### ABBREVIATIONS AND DEFINITIONS

Unless defined otherwise, all technical and scientific terms used herein have the same meaning as is commonly understood by one of ordinary skill in the art to which this disclosure belongs. A comprehensive list of abbreviations utilized by organic chemists (i.e., persons of ordinary skill in the art) appears in the first issue of each volume of the Journal of Organic Chemistry. The list, which is typically presented in a table entitled “Standard List of Abbreviations” is incorporated herein by reference. In the event that there is a plurality of definitions for terms cited herein, those in this section prevail unless otherwise stated.

The following abbreviations and terms have the indicated meanings throughout:
Ac=acetyl
ADC=antibody drug conjugate
Aq=aqueous
Boc=t-butyloxy carbonyl
Bu=butyl
c-=cyclo
DAR=drug-antibody ratio
DCM=dichloromethane=methylene chloride=CH₂Cl₂
DMA=dimethylacetamide
DMF=N,N-dimethylformamide
DPBS=Dulbecco's phosphate buffered saline
eq. Or equiv.=equivalent(s)
Et=ethyl
h=hour(s)
haut=hexafluorophosphate azabenzotriazole tetramethyl uronium
HOBt=hydroxybenzotriazole
IRF=Interferon Regulatory Factor
ma=maleimide acetyl
me=maleimidocaproyl
mCPBA=meta-Chloroperoxybenzoic acid
Me=methyl
min.=minute(s)
MMAE=monomethyl auristatin E
MMAF=monomethyl auristatin F
mp=malieimide propionyl
PAB=4-aminobenzyl
PABC=p-aminobenzylcarbamate
Pg=protecting group
Ph=phenyl
PNGase F=Peptide:N-glycosidase F
PNP=p-nitrophenol
RT=room temperature
sat'd or sat.=saturated
SEAP=secreted embryonic alkaline phosphatase
STD=standard deviation
t- or tert=tertiary
TCEP=tris(2-carboxyethyl)phosphine
TFA=trifluoroacetic acid
THF=tetrahydrofuran
Tosyl=p-toluenesulfonyl
TPPMS=triphenylphosphine meta sulfonic acid
UPLC=ultra performance liquid chromatography

As used herein, the terms “comprising” and “including” or grammatical variants thereof are to be taken as specifying the stated features, integers, steps or components but do not preclude the addition of one or more additional features, integers, steps, components or groups thereof. This term encompasses the terms “consisting of” and “consisting essentially of”.

The phrase “consisting essentially of” or grammatical variants thereof when used herein are to be taken as specifying the stated features, integers, steps or components but do not preclude the addition of one or more additional features, integers, steps, components or groups thereof, but only if the additional features, integers, steps, components or groups thereof do not materially alter the basic and novel characteristics of the claimed composition or method.

For purposes of the present disclosure, the term “antibody” (or “Ab” or “AB”) herein is used in the broadest sense and specifically covers intact monoclonal antibodies, polyclonal antibodies, monospecific antibodies, multispecific antibodies (e.g., bispecific antibodies), genetically engineered forms of the antibodies, and combinations thereof. In certain aspects, the antibody or other such targeting molecule acts to deliver a drug to the particular target cell population with which the antibody or other targeting molecule interacts. In one embodiment, “Ab” comprises an antibody. While some specific examples of antibodies (i.e., “Ab”) are disclosed herein, antibodies that can successfully be used are not limited to these examples, as the person of skill will understand.

The term “antibody,” which is used interchangeably with the term “immunoglobulin,” includes full length (i.e., naturally occurring or formed by normal immunoglobulin gene fragment recombinatorial processes) immunoglobulin molecules (e.g., an IgG antibody). Antibody fragments, which again may be naturally occurring or synthetic in nature, may also be included, provided that both the Q295 location and at least two cysteine payload-accepting sites are present. Accordingly, the term “antibody fragment” includes a portion of an antibody such as F(ab′)₂, F(ab)₂, Fab′, Fab, Fv, scFv and the like. Regardless of structure, an antibody fragment binds with the same antigen that is recognized by the full-length antibody. Methods of making and screening antibody fragments are well-known in the art.

Naturally occurring antibodies typically have two identical heavy chains and two identical light chains, with each light chain covalently linked to a heavy chain by an inter-chain disulfide bond and multiple disulfide bonds further link the two heavy chains to one another. Individual chains may fold into domains having similar sizes (110-125 amino acids) and structures, but different functions. The light chain can comprise one variable domain (V_{L}) and/or one constant domain (CL). The heavy chain can also comprise one variable domain (V_{H}) and/or, depending on the class or isotype of antibody, three or four constant domains (CH1, CH2, CH3, and CH4). The variable region binds to and interacts with a target antigen. The variable region includes a complementary determining region (CDR) that recognizes and binds to a specific binding site on a particular antigen. The constant region may be recognized by and interact with the immune system (see, e.g., Janeway et al., IMMUNOBIOLOGY, 5th Ed., Garland Science (New York 2001), which is hereby incorporated by reference in its entirety). An antibody can be of any type or class (e.g., IgG, IgE, IgM, IgD, and IgA) or subclass (e.g., IgG1, IgG2, IgG3, IgG4, IgAQ1, and IgA2). In humans, the isotypes are IgA, IgD, IgE, IgG, and IgM, with IgA and IgG further subdivided into subclasses or subtypes (IgAQ1-2 and IgG1-4). The antibody can be derived from any suitable species. In some embodiments, the antibody is of human or murine origin. An antibody can be, for example, human, humanized or chimeric.

Generally, the variable domains show considerable amino acid sequence variability from one antibody to the next, particularly at the location of the antigen-binding site. Three regions, called hyper-variable or complementarity-determining regions (CDRs), are found in each of V_{L} and V_{H}, which are supported by less variable regions called framework variable regions. Antibodies include IgG monoclonal antibodies as well as antibody fragments or engineered forms. These are, for example, Fv fragments, or proteins wherein the CDRs and/or variable domains of the exemplified antibodies are engineered as single-chain antigen-binding proteins.

Single chain antibodies lack some or all of the constant domains of the whole antibodies from which they are derived. Therefore, they can overcome some of the problems associated with the use of whole antibodies. For example, single-chain antibodies tend to be free of certain undesired interactions between heavy-chain constant regions and other biological molecules. Additionally, single-chain antibodies are considerably smaller than whole antibodies and can have greater permeability than whole antibodies, allowing single-chain antibodies to localize and bind to target antigen-binding sites more efficiently. Furthermore, the relatively small size of single-chain antibodies makes them less likely to provoke an unwanted immune response in a recipient than whole antibodies.

Fab (Fragment, antigen binding) refers to the fragments of the antibody consisting of the V_{L}, CL, V_{H}, and CH1 domains. Those generated following papain digestion simply are referred to as Fab and do not retain the heavy chain hinge region. Following pepsin digestion, various Fabs retaining the heavy chain hinge are generated. Those fragments with the interchain disulfide bonds intact are referred to as F(ab′)₂, while a single Fab′ results when the disulfide bonds are not retained. F(ab′)₂ fragments have higher avidity for antigen that the monovalent Fab fragments.

Fc (Fragment crystallization) is the designation for the portion or fragment of an antibody that comprises paired heavy chain constant domains. In an IgG antibody, for example, the Fc comprises CH2 and CH3 domains. The Fc of an IgA or an IgM antibody further comprises a CH4 domain. The Fc is associated with Fc receptor binding, activation of complement mediated cytotoxicity and antibody-dependent cellular-cytotoxicity (ADCC). For antibodies such as IgA and IgM, which are complexes of multiple IgG-like proteins, complex formation requires Fc constant domains.

Finally, the hinge region separates the Fab and Fc portions of the antibody, providing for mobility of Fabs relative to each other and relative to Fc, as well as including multiple disulfide bonds for covalent linkage of the two heavy chains.

Antibody “specificity” refers to selective recognition of an antibody for a particular epitope of an antigen. The term “epitope” includes any protein determinant capable of specific binding to an immunoglobulin or T-cell receptor or otherwise interacting with a molecule. Epitopic determinants generally consist of chemically active surface groupings of molecules such as amino acids or carbohydrate or sugar side chains and generally have specific three-dimensional structural characteristics, as well as specific charge characteristics. An epitope may be “linear” or “conformational.” In a linear epitope, all of the points of interaction between the protein and the interacting molecule (such as an antibody) occur linearly along the primary amino acid sequence of the protein. In a conformational epitope, the points of interaction occur across amino acid residues on the protein that are separated from one another, i.e., noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen. As described herein, the phrases “specifically binds” and “specific binding” refer to antibody binding to a predetermined antigen.

Useful polyclonal antibodies are heterogeneous populations of antibody molecules derived from the sera of immunized animals. Useful monoclonal antibodies are homogeneous populations of antibodies to a particular antigenic determinant (e.g., a cancer cell antigen, a viral antigen, a microbial antigen, a protein, a peptide, a carbohydrate, a chemical, nucleic acid, or fragments thereof). A monoclonal antibody (mAb) to an antigen-of-interest can be prepared by using any technique known in the art which provides for the production of antibody molecules by continuous cell lines in culture.

The term “monoclonal antibody” as used herein refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. The modifier “monoclonal” indicates the character of the antibody as being obtained from a substantially homogeneous population of antibodies, and is not to be construed as requiring production of the antibody by any particular method.

Monoclonal antibodies may be murine, human, humanized, or chimeric. A humanized antibody is a recombinant protein in which the CDRs of an antibody from one species; e.g., a rodent, rabbit, dog, goat, horse, or chicken antibody (or any other suitable animal antibody), are transferred into human heavy and light variable domains. The constant domains of an antibody molecule are derived from those of a human antibody. Methods for making humanized antibodies are well known in the art. Chimeric antibodies preferably have constant regions derived substantially or exclusively from human antibody constant regions and variable regions derived substantially or exclusively from the sequence of the variable region from a mammal other than a human. The chimerization process can be made more effective by also replacing the variable regions—other than the hyper-variable regions or the complementarity—determining regions (CDRs), of a murine (or other non-human mammalian) antibody with the corresponding human sequences. The variable regions other than the CDRs are also known as the variable framework regions (FRs).

The term “monoclonal antibodies” specifically includes “chimeric” antibodies in which a portion of the heavy and/or light chain is identical to or homologous with the corresponding sequence of antibodies derived from a particular species or belonging to a particular antibody class or subclass, while the remainder of the chain(s) is identical to or homologous with the corresponding sequences of antibodies derived from another species or belonging to another antibody class or subclass, as well as fragments of such antibodies, so long as they exhibit the desired biological activity.

Useful monoclonal antibodies include, but are not limited to, human monoclonal antibodies, humanized monoclonal antibodies, antibody fragments, or chimeric monoclonal antibodies. Human monoclonal antibodies may be made by any of numerous techniques known in the art (e.g., Teng et al., “Construction and Testing of Mouse—Human Heteromyelomas for Human Monoclonal Antibody Production,” Proc. Natl. Acad. Sci. USA 80:7308-12 (1983); Kozbor et al., “The Production of Monoclonal Antibodies From Human Lymphocytes,” Immunology Today 4:72-79 (1983); and Olsson et al., “Human-Human Monoclonal Antibody-Producing Hybridomas: Technical Aspects,” Meth. Enzymol. 92:3-16 (1982), all of which are hereby incorporated by reference in their entirety).

The antibody can also be a bispecific antibody. Methods for making bispecific antibodies are known in the art and are discussed herein.

An “intact antibody” as described herein includes one which comprises an antigen-binding variable region as well as a light chain constant domain (CL) and heavy chain constant domains, Cm, CH2, Cm and CH4, as appropriate for the antibody class. The constant domains may be native sequence constant domains (e.g., human native sequence constant domains) or amino acid sequence variants thereof.

An intact antibody may have one or more “effector functions,” which refers to those biological activities attributable to the Fc region (e.g., a native sequence Fc region or amino acid sequence variant Fc region) of an antibody. Examples of antibody effector functions include complement dependent cytotoxicity, antibody-dependent cell-mediated cytotoxicity (ADCC) and antibody-dependent cell-mediated phagocytosis. See WO 2014/068443 to Pfizer Inc., which is hereby incorporated by reference in its entirety.

The term “variable” in the context of an antibody refers to certain portions of the variable domains of the antibody that differ extensively in sequence and are used in the binding and specificity of each particular antibody for its particular antigen. This variability is concentrated in three segments called “hypervariable regions” in the light chain and the heavy chain variable domains. The more highly conserved portions of variable domains are called the framework regions (FRs). The variable domains of native heavy and light chains each comprise four FRs connected by three hypervariable regions.

The phrase “hypervariable region” as used herein includes the amino acid residues of an antibody which are responsible for antigen-binding. The hypervariable region generally comprises amino acid residues from a “complementarity determining region” or “CDR” (e.g., residues 24-34 (L1), 50-56 (L2) and 89-97 (L3) in the light chain variable domain and 31-35 (H1), 50-65 (H2) and 95-102 (L3) in the heavy chain variable domain (Kabat et al., SEQUENCES OF PROTEINS OF IMMUNOLOGICAL INTEREST, Fifth Edition, National Institute of Health (Bethesda, Md. 1991), which is hereby incorporated by reference in its entirety); and/or those residues from a “hypervariable loop” (e.g., residues 26-32 (L1), 50-52 (L2) and 91-96 (L3) in the light chain variable domain and 26-32 (H1), 53-55 (142) and 96-101 (H3) in the heavy chain variable domain; Chothia and Lesk, “Canonical Structures For the Hypervariable Regions of Immunoglobulins,” J. Mol. Biol. 196:901-17 (1987), which is hereby incorporated by reference in its entirety). FR residues are those variable domain residues other than the hypervariable region residues as herein defined.

A “single-chain Fv” or “scFv” antibody fragment may include the V.sub.H and V.sub.L domains of an antibody, where these domains are present in a single polypeptide chain. Typically, the Fv polypeptide further comprises a polypeptide linker between the V.sub.H and V.sub.L domains which enables the scFv to form the desired structure for antigen binding. For a review of scFv, see Pluckthun in THE PHARMACOLOGY OF MONOCLONAL ANTIBODIES, vol. 113, Rosenburg and Moore eds., SpringerVerlag (New York 1994) pp. 269-315, which is hereby incorporated by reference in its entirety).

The term “diabody” includes small antibody fragments with two antigen-binding sites, which fragments comprise a variable heavy domain (V_{H}) connected to a variable light domain (V_{L}) in the same polypeptide chain. By using a linker that is too short to allow pairing between the two domains on the same chain, the domains are forced to pair with the complementary domains of another chain and create two antigen-binding sites. Diabodies are described more fully in, for example, EP 0 404 097 to BEHRINGWERKE AG; WO 93/11161 to Enzon, Inc.; and Hollinger et al., “‘Diabodies’: Small Bivalent and Bispecific Antibody Fragments,” Proc. Natl. Acad. Sci. USA 90:6444-6448 (1993), all of which are hereby incorporated by reference in their entirety.

Completely human antibodies are useful and can be produced using transgenic mice that are incapable of expressing endogenous immunoglobulin heavy and light chains genes, but which can express human heavy and light chain genes. The transgenic mice are immunized in the normal fashion with a selected antigen, e.g., all or a portion of a polypeptide of the present disclosure. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, “Human Antibodies From Transgenic Mice,” Int. Rev. Immunol. 13:65-93 (1995), which is hereby incorporated by reference in its entirety. For a detailed discussion of this technology for producing human antibodies and human monoclonal antibodies and protocols for producing such antibodies, see, e.g., U.S. Pat. No. 5,625,126 to Lonberg et al.; U.S. Pat. No. 5,633,425 to Lonberg et al.; U.S. Pat. No. 5,569,825 to Lonberg et al.; U.S. Pat. No. 5,661,016 to Lonberg et al.; U.S. Pat. No. 5,545,806 to Lonberg et al., all of which are hereby incorporated by reference in their entirety.

Completely human antibodies that recognize a selected epitope can be generated using a technique referred to as “guided selection.” In this approach a selected non-human monoclonal antibody, e.g., a mouse antibody, is used to guide the selection of a completely human antibody recognizing the same epitope. See, e.g., Jespers et al., “Guiding the Selection of Human Antibodies From Phage Display Repertoires to a Single Epitope of an Antigen,” Biotechnology 12:899-903 (1994), which is hereby incorporated by reference in its entirety. Human antibodies can also be produced using various techniques known in the art, including phage display libraries (see, e.g., Hoogenboom and Winter, “By-Passing Immunisation. Human Antibodies From Synthetic Repertoires of Germline V_{H} Gene Segments Rearranged In Vitro,” J. Mol. Biol. 227:381 (1991); Marks et al., “By-Passing Immunization. Human Antibodies From V-gene Libraries Displayed on Phage,” J. Mol. Biol. 222:581 (1991); Quan and Carter, “The rise of monoclonal antibodies as therapeutics,” In ANTI-IGE AND ALLERGIC DISEASE, Jardieu and Fick, eds., Marcel Dekker (New York, N.Y., 2002) Chapter 20, pp. 427-469), all of which are hereby incorporated by reference in their entirety.

“Humanized” forms of non-human (e.g., rodent) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody will comprise at least a portion of an immunoglobulin constant region (Fc), typically that of a human immunoglobulin. For further details, see Jones et al., “Replacing the Complementarity-Determining Regions in a Human Antibody With Those From a Mouse,” Nature 321:522-25 (1986); Riechmann et al., “Reshaping Human Antibodies For Therapy,” Nature 332:323-329 (1988); and Presta, L. “Antibody Engineering,” Curr. Op. Struct. Biol. 2:593-596 (1992), all of which are hereby incorporated by reference in their entirety.

Recombinant antibodies, such as chimeric and humanized monoclonal antibodies, comprising both human and non-human portions, which can be made using standard recombinant DNA techniques, are useful antibodies. A chimeric antibody is a molecule in which different portions are derived from different animal species, such as for example, those having a variable region derived from a murine monoclonal and human immunoglobulin constant regions (see, e.g., U.S. Pat. No. 4,816,567 to Cabilly et al.; and U.S. Pat. No. 4,816,397 to Boss et al., which are incorporated herein by reference in their entirety). Humanized antibodies are antibody molecules from non-human species having one or more complementarity determining regions (CDRs) from the non-human species and a framework region from a human immunoglobulin molecule (see, e.g., U.S. Pat. No. 5,585,089 to Queen et al., which is incorporated herein by reference in its entirety). Such chimeric and humanized monoclonal antibodies can be produced by recombinant DNA techniques known in the art, for example using methods described in International Publication No. WO 87/02671 to Int Genetic Eng; European Patent Publication No. 0 184 187 to Teijin Ltd; European Patent Publication No. 0 171 496 to Japan Res Dev Corp; European Patent Publication No. 0 173 494 to Univ Leland Stanford Junior; International Publication No. WO 86/01533 to Celltech Ltd; U.S. Pat. No. 4,816,567 to Cabilly et al.; Berter et al., “Escherichia coli Secretion of an Active Chimeric Antibody Fragment,” Science 240:1041-1043 (1988); Liu et al., “Chimeric Mouse-Human IgG1 Antibody That Can Mediate Lysis of Cancer Cells,” Proc. Natl. Acad. Sci. USA 84:3439-3443 (1987); Liu et al., “Production of a Mouse-Human Chimeric Monoclonal Antibody to CD20 With Potent Fc-Dependent Biologic Activity,” J. Immunol. 139:3521-3526 (1987); Sun et al., “Chimeric Antibody With Human Constant Regions and Mouse Variable Regions Directed Against Carcinoma-Associated Antigen 17-1A,” Proc. Natl. Acad. Sci. USA 84:214-218 (1987); Nishimura et al., “Recombinant Human-Mouse Chimeric Monoclonal Antibody Specific for Common Acute Lymphocytic Leukemia Antigen,” Cancer. Res. 47:999-1005 (1987); Wood et al., “The Synthesis and In Vivo Assembly of Functional Antibodies in Yeast,” Nature 314:446-449 (1985); and Shaw et al., “Mouse/Human Chimeric Antibodies to a Tumor-Associated Antigen: Biologic Activity of the Four Human IgG Subclasses,” J. Natl. Cancer Inst. 80:1553-1559 (1988); Morrison, S. L., “Transfectomas Provide Novel Chimeric Antibodies,” Science 229:1202-1207 (1985); U.S. Pat. No. 5,225,539 to Winter; Jones et al., “Replacing the Complementarity-Determining Regions in a Human Antibody With Those From a Mouse,” Nature 321:552-525 (1986); Verhoeyan et al., “Reshaping Human Antibodies: Grafting an Antilysozyme Activity,” Science 239:1534 (1988); and Beidler et al., “Cloning and High Level Expression of a Chimeric Antibody With Specificity For Human Carcinoembryonic Antigen,” J. Immunol. 141:4053-4060 (1988), all of which are hereby incorporated by reference in their entirety.

As described herein, “isolated” includes separated from other components of (a) a natural source, such as a plant or animal cell or cell culture, or (b) a synthetic organic chemical reaction mixture. As used herein, “purified” means that when isolated, the isolate contains at least 95%, and in another aspect at least 98%, of a compound (e.g., a conjugate) by weight of the isolate.

An “isolated” antibody is one which has been identified and separated and/or recovered from component of its natural environment. Contaminant components of its natural environment are materials which would interfere with diagnostic or therapeutic uses for the antibody, and may include enzymes, hormones, and other proteinaceous or non-proteinaceous solutes. In some embodiments, the antibody may be purified (1) to greater than 95% by weight of antibody as determined by the Lowry method, and in some embodiments more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody may include the antibody in situ within recombinant cells since at least one component of the antibody's natural environment will not be present. Ordinarily, an isolated antibody may be prepared by at least one purification step.

An antibody which “induces apoptosis” is one which induces programmed cell death as determined by binding of annexin V, fragmentation of DNA, cell shrinkage, dilation of endoplasmic reticulum, cell fragmentation, and/or formation of membrane vesicles (called apoptotic bodies). The cell may be a tumor cell, e.g., a breast, ovarian, stomach, endometrial, salivary gland, lung, kidney, colon, thyroid, pancreatic or bladder cell. Various methods are available for evaluating the cellular events associated with apoptosis. For example, phosphatidyl serine (PS) translocation can be measured by annexin binding; DNA fragmentation can be evaluated through DNA laddering; and nuclear/chromatin condensation along with DNA fragmentation can be evaluated by any increase in hypodiploid cells.

In other embodiments, the antibody is a fusion protein of an antibody, or a functionally active fragment thereof, for example in which the antibody is fused via a covalent bond (e.g., a peptide bond), at either the N-terminus or the C-terminus to an amino acid sequence of another protein (or portion thereof, preferably at least 10, 20 or 50 amino acid portion of the protein) that is not from an antibody. In one embodiment, the antibody or fragment thereof is covalently linked to the other protein at the N-terminus of the constant domain.

Antibodies include analogs and derivatives that are either modified, i.e., by the covalent attachment of any type of molecule as long as such covalent attachment permits the antibody to retain its antigen binding immunospecificity. For example, derivatives and analogs of the antibodies include those that have been further modified, e.g., by glycosylation, acetylation, pegylation, phosphorylation, amidation, derivatization by known protecting/blocking groups, proteolytic cleavage, linkage to a cellular antibody unit or other protein. Any of numerous chemical modifications can be carried out by known techniques including, but not limited to, specific chemical cleavage, acetylation, formylation, and metabolic synthesis in the presence of tunicamycin. Additionally, the analog or derivative may contain one or more unnatural amino acids.

Antibodies may have modifications (e.g., substitutions, deletions or additions) in amino acid residues that interact with Fc receptors. In particular, antibodies may have modifications in amino acid residues identified as involved in the interaction between the anti-Fc domain and the FcRn receptor (see, e.g., International Publication No. WO 97/34631, which is incorporated herein by reference in its entirety).

In one embodiment, Ab (i.e., the antibody) is a tumor targeting antibody, a bispecific antibody, a monoclonal antibody, a chimeric antibody, or a humanized antibody. Antibody fragments may also be included, provided that both the Q295 location and at least two cysteine payload-accepting sites are present.

Antibodies immunospecific for a cancer cell antigen can be obtained commercially or produced by any method known to one of skill in the art such as, e.g., chemical synthesis or recombinant expression techniques. The nucleotide sequence encoding antibodies immunospecific for a cancer cell antigen can be obtained, e.g., from the GenBank database or a database like it, literature publications, or by routine cloning and sequencing.

In one embodiment, Ab (i.e., the antibody) is selected from the group consisting of anti-Her2 antibody, anti-CD20 antibody, anti-CD38 antibody, anti-IL-6 receptor antibody, anti-VEGRF2 antibody, anti-HER-2 antibody, anti-DLL3 antibody, anti-Nectin4 antibody, anti-CD33 antibody, anti-CD79b antibody, anti-CD11a antibody, anti-BCMA antibody, anti-CD22 antibody, anti-Trop2 antibody, anti-FRα antibody, anti-EpCAM antibody, anti-mesothelin antibody, anti-LIV1 antibody, oregovomab, edrecolomab, cetuximab, a humanized monoclonal antibody to the vitronectin receptor (αᵥβ₃), alemtuzumab, a humanized anti-HLA-DR antibody for the treatment of non-Hodgkin's lymphoma, 1311 Lym-1, a murine anti-HLA-Dr10 antibody for the treatment of non-Hodgkin's lymphoma, a humanized anti-CD2 mAb for the treatment of Hodgkin's Disease or non-Hodgkin's lymphoma, labetuzumab, bevacizumab, ibritumomab tiuxetan, ofatumumab, panitumumab, rituximab, tositumomab, ipilimumab, gemtuzumab, humanized monoclonal antibody to the oncofecal protein receptor 5T4, M1/70 (antibody to CD11b receptor), anti-MRC1, anti GCC, anti CD32, and other antibodies.

In one embodiment, known antibodies for the treatment of cancer may be used. Antibodies immunospecific for a cancer cell antigen can be obtained commercially or produced by any method known to one of skill in the art such as, e.g., recombinant expression techniques. The nucleotide sequence encoding antibodies immunospecific for a cancer cell antigen can be obtained, e.g., from the GenBank database or a database like it, the literature publications, or by routine cloning and sequencing. Examples of antibodies available for the treatment of cancer include, but are not limited to, Oregovomab or OVAREX® which is a murine antibody for the treatment of ovarian cancer; Edrecolomab or panorex which is a murine IgG2a antibody for the treatment of colorectal cancer; Cetuximab (e.g., ERBITUX©) which is an anti-EGFR IgG chimeric antibody for the treatment of epidermal growth factor positive cancers, such as head and neck cancer; vitaxin, which is a humanized antibody for the treatment of sarcoma; Alemtuzumab or CAMPATH-1H, which is a humanized IgG1 antibody for the treatment of chronic lymphocytic leukemia (CLL); ONCOLYM, which is a radio labeled murine anti-HLA-Dr10 antibody for the treatment of non-Hodgkin's lymphoma; ALLOMUNE (Bio Transplant, CA) which is a humanized anti-CD2 mAb for the treatment of Hodgkin's Disease or non-Hodgkin's lymphoma; and CEA-Cide (Immunomedics, NJ) which is a humanized anti-CEA antibody for the treatment of colorectal cancer.

The terms “protein,” “polypeptide,” and “peptide” may be referred to interchangeably herein. The terms may be distinguished as follows. A protein typically refers to the end product of transcription, translation, and post-translation modifications in a cell.

A polypeptide may include a protein or a peptide. A peptide, in contrast to a protein, typically is a short polymer of amino acids, of a length typically of 100 or less amino acids.

The term “peptide” or “polypeptide” as used herein refers to proteins and fragments thereof. Peptides may include amino acid sequences. Those sequences may be written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Citrulline (Cit), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

The peptides of the immunotherapy compounds may be derived from nature, or may, alternatively be designed de nova. A peptide is said to be “derivable from a naturally occurring amino acid sequence” if it can be obtained by fragmenting a naturally occurring sequence, or if it can be synthesized based upon knowledge of the sequence of the naturally occurring amino acid sequence or of the genetic material (DNA or RNA) that encodes this sequence.

The peptides of the immunotherapy compounds may or may not share substantial homology or identity with naturally occurring proteins or portions thereof (e.g., peptides). The immunotherapy compound may or may not include peptides with “substantial similarity” with naturally occurring proteins or portions thereof (e.g., peptides). A peptide with substantial similarity includes peptides with at least 70% or greater sequence homology or identity with a peptide having the same number of amino acid residues as the reference peptide. In some instances, a peptide with substantial similarity includes peptides with at least 75% or greater, or 80% or greater, or 85% or greater, or 90% or greater, or 92% or greater, or 95% or greater, or 97% or greater, or 99% or greater sequence homology or identity with a peptide having the same number of amino acid residues as the reference peptide.

The terms loading or “drug loading” or “payload loading” refer to the average number of payloads (“payload” and “payloads” are used interchangeably herein with “drug” and “drugs”) per antibody in an ADC molecule. Drug loading may range from 1 to 50 drugs per antibody. This is sometimes referred to as the DAR, or drug to antibody ratio. Compositions of the ADCs described herein typically have DAR's of from 1-25, and in certain embodiments, from 1-8, from 2-8, from 2-6, from 2-5 and from 2-4. Typical DAR values include 2, 4, 6, 8, and 10. The average number of drugs per antibody, or DAR value, may be characterized by conventional means such as UV/visible spectroscopy, mass spectrometry, ELISA assay, and HPLC. The quantitative DAR value may also be determined. In some instances, separation, purification, and characterization of homogeneous ADCs having a particular DAR value may be achieved by means such as reverse phase HPLC or electrophoresis. DAR may be limited by the number of attachment sites on the antibody. For example, where the attachment is a cysteine thiol, an antibody may have only one or several cysteine thiol groups, or may have only one or several sufficiently reactive thiol groups through which a linker unit may be attached. In some embodiments, the cysteine thiol is a thiol group of a cysteine residue that forms an interchain disulfide bond. In some embodiments, the cysteine thiol is a thiol group of a cysteine residue that does not form an interchain disulfide bond. Typically, fewer than the theoretical maximum of drug moieties are conjugated to an antibody during a conjugation reaction. An antibody may contain, for example, many lysine residues that do not react with a linker or linker intermediate. Only the most reactive lysine groups may react with a reactive linker reagent.

Generally, antibodies do not contain many, if any, free and reactive cysteine thiol groups which may be linked to a drug via a linker. Most cysteine thiol residues in the antibodies exist as disulfide bridges and must be reduced with a reducing agent such as dithiothreitol (DTT). The antibody may be subjected to denaturing conditions to reveal reactive nucleophilic groups such as lysine or cysteine. The loading (drug/antibody ratio) of an ADC may be controlled in several different manners, including: (i) limiting the molar excess of drug-linker relative to the antibody, (ii) limiting the conjugation reaction time or temperature, and (iii) partial or limiting reductive conditions for cysteine thiol modification. Where more than one nucleophilic group reacts with a drug-linker then the resulting product is a mixture of ADCs with a distribution of one or more drugs moieties per antibody. The average number of drugs per antibody may be calculated from the mixture by, for example, dual ELISA antibody assay, specific for antibody and specific for the drug. Individual ADCs may be identified in the mixture by mass spectroscopy, and separated by HPLC, e.g., hydrophobic interaction chromatography.

In one embodiment, the antibody may be selected from trastuzumab and a trastuzumab mutant. In some embodiments, the antibody bound via an Fc-containing or Fab-containing polypeptide engineered with an acyl donor glutamine-containing tag (e.g., Gln-containing peptide tags or Q-tags) or an endogenous glutamine made reactive (i.e., the ability to form a covalent bond as an acyl donor in the presence of an amine and a transglutaminase) by polypeptide engineering (e.g., via amino acid deletion, insertion, substitution, mutation, or any combination thereof on the polypeptide), in the presence of transglutaminase.

In certain embodiments, the present disclosure relates to any of the aforementioned antibody drug conjugates and attendant definitions, wherein the antibody drug conjugate comprises between 2, 3, 4, 5, 6, 7, 8, 9, 10, 15, 20, or 25 compounds of the present disclosure, or any number of compounds therein.

In certain embodiments, the present disclosure relates to any of the aforementioned antibody drug conjugates and attendant definitions, wherein the antibody drug conjugate comprises 3 or 4 compounds of the present disclosure.

An amino acid “derivative” includes an amino acid having substitutions or modifications by covalent attachment of a parent amino acid, such as, e.g., by alkylation, glycosylation, acetylation, phosphorylation, and the like. Further included within the contemplated meaning of “derivative” is, for example, one or more analogs of an amino acid with substituted linkages, as well as other modifications known in the art.

A “natural amino acid” refers to arginine, glutamine, phenylalanine, tyrosine, tryptophan, lysine, glycine, alanine, histidine, serine, proline, glutamic acid, aspartic acid, threonine, cysteine, methionine, leucine, asparagine, isoleucine, and valine, unless otherwise indicated by context.

A linker (sometimes referred to as “[linker]” herein) is a bifunctional compound which can be used to link a drug and an antibody to form an antibody drug conjugate (ADC). Such conjugates are useful, for example, in the formation of immunoconjugates directed against tumor associated antigens. Such conjugates may, in some embodiments, allow for the selective delivery of cytotoxic drugs to tumor cells.

A self-immolative spacer as described herein includes covalent assemblies tailored to correlate the cleavage of two chemical bonds after activation of a protective part in a precursor: Upon stimulation, the protective moiety is removed, which generates a cascade of disassembling reactions leading to the temporally sequential release of smaller molecules. See Alouane et al., “Self-Immolative Spacers: Kinetic Aspects, Structure-Property Relationships, and Applications,” Angewandte Chemie 54(26):7492-7509 (2015), which is hereby incorporated by reference in its entirety. Self-immolative spacers were created to address limitations for drug delivery, and have gained wide interest in medicinal chemistry, analytical chemistry, and material science. See Alouane et al., “Self-Immolative Spacers: Kinetic Aspects, Structure-Property Relationships, and Applications,” Angewandte Chemie 54(26:7492-7509 (2015), which is hereby incorporated by reference in its entirety.

The phrase “substantial amount” includes a majority, i.e., greater than 50% of a population, of a mixture or a sample.

The term “intracellular metabolite” refers to a compound resulting from a metabolic process or reaction inside a cell on an antibody-drug conjugate (ADC). The metabolic process or reaction may be an enzymatic process such as proteolytic cleavage of a peptide linker of the ADC. Intracellular metabolites include, but are not limited to, antibodies and free drug which have undergone intracellular cleavage after entry, diffusion, uptake, or transport into a cell.

The terms “intracellularly cleaved” and “intracellular cleavage” refer to a metabolic process or reaction inside a cell on an ADC or the like, whereby the covalent attachment, e.g., the linker, between the drug moiety and the antibody is broken, resulting in the free drug, or other metabolite of the conjugate dissociated from the antibody inside the cell. The cleaved moieties of the ADC are thus intracellular metabolites.

The term “bioavailability” refers to the systemic availability (i.e., blood/plasma levels) of a given amount of a drug administered to a patient. Bioavailability indicates measurement of both the time (rate) and total amount (extent) of drug that reaches the general circulation from an administered dosage form.

The term “cytotoxic activity” refers to a cell-killing, a cytostatic or an anti-proliferative effect of an ADC or an intracellular metabolite of said ADC. Cytotoxic activity may be expressed as the IC50 value, which is the concentration (molar or mass) per unit volume at which half the cells survive.

A “disorder” is any condition that would benefit from treatment with a drug or antibody-drug conjugate. This includes chronic and acute disorders or diseases including those pathological conditions which predispose a mammal to the disorder in question. Non-limiting examples of disorders to be treated herein include benign and malignant cancers; leukemia and lymphoid malignancies, neuronal, glial, astrocytal, hypothalamic and other glandular, macrophagal, epithelial, stromal and blastocoelic disorders; and inflammatory, angiogenic and immunologic disorders.

The terms “cancer” and “cancerous” refer to or describe the physiological condition or disorder in mammals that is typically characterized by unregulated cell growth. A “tumor” comprises one or more cancerous cells.

As used herein, the terms “cell,” “cell line,” and “cell culture” are used interchangeably and all such designations include progeny. The words “transformants” and “transformed cells” include the primary subject cell and cultures or progeny derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

A “patient,” as used herein, includes both humans and other animals, particularly mammals. Thus, the methods are applicable to both human therapy and veterinary applications. Examples of a “patient” include, but are not limited to, a human, rat, mouse, guinea pig, monkey, pig, goat, cow, horse, dog, cat, bird, and fowl. In some embodiments, the patient is a mammal, for example, a primate. In some embodiments, the patient is a human. In one embodiment, the patient is an infant, a juvenile, or an adult.

The terms “treat” or “treatment,” unless otherwise indicated by context, refer to therapeutic treatment and prophylactic measures to prevent relapse, wherein the object is to inhibit or slow down (lessen) an undesired physiological change or disorder, such as the development or spread of cancer or a viral infection.

For purposes of the present disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already having the condition or disorder as well as those prone to have the condition or disorder.

In the context of cancer, the term “treating” includes any or all of inhibiting growth of tumor cells, cancer cells, or of a tumor; inhibiting replication of tumor cells or cancer cells; lessening of overall tumor burden or decreasing the number of cancerous cells; and ameliorating one or more symptoms associated with the disease.

Treatment can involve administering a compound described herein to a patient diagnosed with a disease, and may involve administering the compound to a patient who does not have active symptoms. Conversely, treatment may involve administering the compositions to a patient at risk of developing a particular disease, or to a patient reporting one or more of the physiological symptoms of a disease, even though a diagnosis of this disease may not have been made.

The terms “administer,” “administering” or “administration” in reference to a dosage form of the invention refers to the act of introducing the dosage form into the system of subject in need of treatment. When a dosage form of the invention is given in combination with one or more other active agents (in their respective dosage forms), “administration” and its variants are each understood to include concurrent and/or sequential introduction of the dosage form and the other active agents. Administration of any of the described dosage forms includes parallel administration, co-administration or sequential administration. In some situations, the therapies are administered at approximately the same time, e.g., within about a few seconds to a few hours of one another.

A “therapeutically effective” amount of the compounds described herein is typically one which is sufficient to achieve the desired effect and may vary according to the nature and severity of the disease condition, and the potency of the compound. It will be appreciated that different concentrations may be employed for prophylaxis than for treatment of an active disease. A therapeutic benefit is achieved with the amelioration of one or more of the physiological symptoms associated with the underlying disorder such that an improvement is observed in the patient, notwithstanding that the patient may still be afflicted with the underlying disorder. In the case of cancer, a therapeutically effective amount of a drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may inhibit the growth of and/or kill existing cancer cells, it may be cytostatic and/or cytotoxic. For cancer therapy, efficacy can, for example, be measured by assessing the time to disease progression (TTP) and/or determining the response rate (RR).

As such, the therapeutic effect can be a decrease in the severity of symptoms associated with the disorder and/or inhibition (partial or complete) of progression of the disorder, or improved treatment, healing, prevention or elimination of a disorder, or side-effects. The amount needed to elicit the therapeutic response can be determined based on the age, health, size, and sex of the subject. Optimal amounts can also be determined based on monitoring of the subject's response to treatment. The term “treatment” or “treat” may include effective inhibition, suppression or cessation of symptoms so as to prevent or delay the onset, retard the progression, or ameliorate the symptoms of a condition.

Throughout this specification the terms and substituents retain their definitions. Substituents (e.g., Rⁿ) are generally defined when introduced and retain that definition throughout the specification and in all independent claims.

C₁ to C₂₀ hydrocarbon includes alkyl, cycloalkyl, polycycloalkyl, alkenyl, alkynyl, aryl, and combinations thereof, containing from 1 to 20 carbon atoms, inclusive. Non-limiting examples include ethyl, benzyl, phenethyl, cyclohexylmethyl, camphoryl and naphthylethyl. Hydrocarbon refers to any substituent comprised of hydrogen and carbon as the only elemental constituents.

Alkyl is a subset of hydrocarbon. Unless otherwise specified, alkyl (or alkylene) is intended to include linear or branched saturated hydrocarbon structures and combinations thereof. In some embodiments, alkyl refers to alkyl groups from 1 to 20 carbon atoms, or from 1 to 10 carbon atoms, or from 1 to 8 carbon atoms, or from 1 to 6 carbon atoms, or from 1 to 5 carbon atoms, or from 1 to 4 carbon atoms. Examples of alkyl groups include methyl, ethyl, propyl, isopropyl, n-butyl, s-butyl, t-butyl and the like.

Cycloalkyl is a subset of hydrocarbon and includes cyclic hydrocarbon groups of from 3 to 8 carbon atoms. Examples of cycloalkyl groups include cy-propyl, cy-butyl, cy-pentyl, norbornyl and the like.

Arylalkyl describes an alkyl residue in which a hydrogen atom is substituted by an aryl moiety. C₁ to C₄ (or C₁-C₄, or C₁₄) arylalkyl includes, as non-limiting examples, a phenyl ring attached through a methyl, C₂, C₃, or C₄ alkyl linker, wherein the alkyl may be straight chain or branched.

Oxaalkyl refers to alkyl residues in which one or more carbons (and their associated hydrogens) have been replaced by oxygen. Examples include methoxypropoxy, hydroxymethyl, hydroxyethyl, 3,6,9-trioxadecyl and the like. The term oxaalkyl is intended as it is understood in the art [see Naming and Indexing of Chemical Substances for Chemical Abstracts, published by the American Chemical Society, ¶196, but without the restriction of ¶127(a)], i.e. it refers to compounds in which the oxygen is bonded via a single bond to its adjacent atoms (forming ether bonds); it does not refer to doubly bonded oxygen, as would be found in carbonyl groups. Similarly, azaalkyl refers to alkyl residues in which one or more carbons has been replaced by nitrogen. Examples include ethylaminoethyl and methylaminopropyl, and the like.

Alkoxy or alkoxyl is a subset of oxaalkyl and refers to groups of from 1 to 20 carbon atoms attached to the parent structure through an oxygen. In some embodiments, alkyl refers to alkyl groups from 1 to 20 carbon atoms, or from 1 to 10 carbon atoms, or from 1 to 6 carbon atoms, or from 1 to 5 carbon atoms, or from 1 to 4 carbon atoms of a straight or branched configuration Examples include methoxy, ethoxy, propoxy, isopropoxy and the like. Lower-alkoxy refers to groups containing one to four carbons. For the purpose of this application, alkoxy and lower alkoxy include methylenedioxy and ethylenedioxy.

Aryl and heteroaryl mean (i) a phenyl group (or benzene) or a monocyclic 5-or 6-membered heteroaromatic ring containing 1-4 heteroatoms selected from O, N, or S; (ii) a bicyclic 9-or 10-membered aromatic or heteroaromatic ring system containing 0-4 heteroatoms selected from O, N, or S; or (iii) a tricyclic 13-or 14-membered aromatic or heteroaromatic ring system containing 0-5 heteroatoms selected from O, N, or S. The aromatic 6-to 14-membered carbocyclic rings include, e.g., benzene, naphthalene, indane, tetralin, and fluorene and the 5-to 10-membered aromatic heterocyclic rings include, e.g., imidazole, pyridine, indole, thiophene, benzopyranone, thiazole, furan, benzimidazole, quinoline, isoquinoline, quinoxaline, pyrimidine, pyrazine, tetrazole and pyrazole. As used herein aryl and heteroaryl refer to residues in which one or more rings are aromatic, but not all need be. In some embodiments, aryl refers to a phenyl group. In some embodiments, heteroaryl refers to pyridine, imidazole, pyrimidine, indole, thiophene, benzopyranone, thiazole, furan, benzimidazole, quinoline, isoquinoline, quinoxaline, pyrimidine, pyrazine, tetrazole and pyrazole. In other embodiments, heteroaryl refers to pyridine, pyridazine, pyrazine, or pyrimidine. In still other embodiments, heteroaryl refers to pyridine.

Heterocycle means a cycloalkyl or aryl carbocycle residue in which from one to four carbons is replaced by a heteroatom selected from the group consisting of N, O and S. The nitrogen and sulfur heteroatoms may optionally be oxidized, and the nitrogen heteroatom may optionally be quaternized. Unless otherwise specified, a heterocycle may be non-aromatic or aromatic. Non-limiting examples of heterocycles that fall within the scope of the invention include pyrrolidine, pyrazole, pyrrole, indole, quinoline, isoquinoline, tetrahydroisoquinoline, benzofuran, benzodioxan, benzodioxole (commonly referred to as methylenedioxyphenyl, when occurring as a substituent), tetrazole, morpholine, thiazole, pyridine, pyridazine, pyrimidine, thiophene, furan, oxazole, oxazoline, isoxazole, dioxane, tetrahydrofuran and the like. It is to be noted that heteroaryl is a subset of heterocycle in which the heterocycle is aromatic. Examples of heterocyclyl residues additionally include piperazinyl, 2-oxopiperazinyl, 2-oxopiperidinyl, 2-oxo-pyrrolidinyl, 2-oxoazepinyl, azepinyl, 4-piperidinyl, pyrazolidinyl, imidazolyl, imidazolinyl, imidazolidinyl, pyrazinyl, oxazolidinyl, isoxazolidinyl, thiazolidinyl, isothiazolyl, quinuclidinyl, isothiazolidinyl, benzimidazolyl, thiadiazolyl, benzopyranyl, benzothiazolyl, tetrahydrofuryl, tetrahydropyranyl, thienyl, benzothienyl, thiamorpholinyl, thiamorpholinylsulfoxide, thiamorpholinylsulfone, oxadiazolyl, triazolyl and tetrahydroquinolinyl.

A nitrogen heterocycle is a heterocycle containing at least one nitrogen in the ring; it may contain additional nitrogens, as well as other heteroatoms. Non-limiting examples include piperidine, piperazine, morpholine, pyrrolidine and thiomorpholine. Nitrogen heteroaryl is a subset of nitrogen heterocycle; examples include pyridine, pyrrole and thiazole.

The term “halogen” means fluorine, chlorine, bromine or iodine atoms. In one embodiment, halogen may be a fluorine or chlorine atom.

Unless otherwise specified, acyl refers to formyl and to groups of 1, 2, 3, 4, 5, 6, 7 and 8 carbon atoms of a straight, branched, cyclic configuration, saturated, unsaturated and aromatic and combinations thereof, attached to the parent structure through a carbonyl functionality. Examples include acetyl, benzoyl, propionyl, isobutyryl and the like. Lower-acyl refers to groups containing one to four carbons. The double bonded oxygen, when referred to as a substituent itself is called “oxo.”

As used herein, the term “optionally substituted” may be used interchangeably with “unsubstituted or substituted.” The term “substituted” refers to the replacement of one or more hydrogen atoms in a specified group with a specified radical. For example, “substituted aryl” or “substituted heteroaryl” refers to aryl or heteroaryl wherein one or more H atoms in each residue are replaced with halogen, haloalkyl, alkyl, alkoxy, or haloalkoxy.

The compounds described herein may contain asymmetric centers and may thus give rise to enantiomers, diastereomers, and other stereoisomeric forms which may be defined in terms of absolute stereochemistry as (R)- or (S)-. The present invention is meant to include all such possible diastereomers as well as their racemic and optically pure forms. Optically active (R)-and (S)-isomers may be prepared using homo-chiral synthons or homo-chiral reagents, or optically resolved using conventional techniques. When the compounds described herein contain olefinic double bonds or other centers of geometric asymmetry, and unless specified otherwise, it is intended to include both (E)-and (Z)-geometric isomers. Likewise, all tautomeric forms are intended to be included.

The graphic representations of racemic, ambiscalemic and scalemic or enantiomerically pure compounds used herein are a modified version of the denotations taken from Maehr J. Chem. Ed. 62, 114-120 (1985): simple lines provide no information about stereochemistry and convey only connectivity; solid and broken wedges are used to denote the absolute configuration of a chiral element; solid and broken bold lines are geometric descriptors indicating the relative configuration shown but not necessarily denoting racemic character; and wedge outlines and dotted or broken lines denote enantiomerically pure compounds of indeterminate absolute configuration. For example, the graphic representation

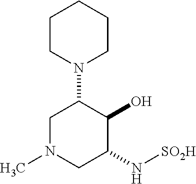

indicates either, or both, of the two trans:trans enantiomers:

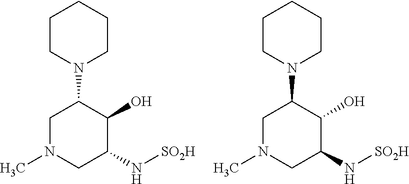

in any ratio, from pure enantiomers to racemates. The graphic representation:

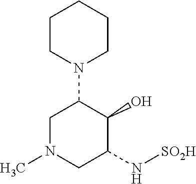

indicates a single enantiomer of unknown absolute stereochemistry, i.e., it could be either of the two preceding structures, as a substantially pure single enantiomer. And, finally, the representation:

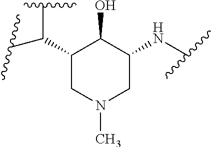 [SMILES: *N[C@@H]1CN(C)C[C@H](C(*)*)[C@H]1O]

indicates a pure (R,R,S) absolute configuration. For the purpose of the present disclosure, a “pure” or “substantially pure” enantiomer is intended to mean that the enantiomer is at least 95% of the configuration shown and 5% or less of other enantiomers. Similarly, a “pure” or “substantially pure” diastereomer is intended to mean that the diastereomer is at least 95% of the relative configuration shown and 5% or less of other diastereomers. In some embodiments, the purity of the compound is at least 99%.

In any of these possibilities, compounds can be a single stereoisomer or a mixture. If a mixture, the mixture will most commonly be racemic, but it need not be. Substantially pure single stereoisomers of biologically active compounds such as those described herein often exhibit advantages over their racemic mixture.

Enantiomerically pure means greater than 80 e.e., and preferably greater than 90 e.e. For the purpose of the present disclosure, a “pure” or “substantially pure” stereoisomer is intended to mean that the stereoisomer is at least 95% of the configuration shown and 5% or less of other stereoisomers, or at least 97% of the configuration shown and 3% or less of other stereoisomers, or at least 99% of the configuration shown and 1% or less of other stereoisomers.

It may be found upon examination that certain species and genera are not patentable to the inventors in this application. In this case, the exclusion of species and genera in applicants' claims are to be considered artifacts of patent prosecution and not reflective of the inventors' concept or description of their invention, which encompasses all members of the genus that are not in the public's possession.

As used herein, the recitation of “compound” may also be used in reference to an “antibody-drug conjugate”—unless expressly further limited. In some embodiments, the term “compound of formula” has the same meaning as “antibody-drug conjugate of formula,” and refers to antibody-drug conjugate, or a pharmaceutically acceptable salt thereof.

As used herein, and as would be understood by the person of skill in the art, the recitation of “compound” or “antibody-drug conjugate”—unless expressly further limited—is intended to include salts of that compound or antibody-drug conjugate. In a particular embodiment, the term “compound of formula” or “antibody-drug conjugate of formula” refers to the compound or antibody-drug conjugate, or a pharmaceutically acceptable salt thereof.

The term “pharmaceutically acceptable salt” refers to salts prepared from pharmaceutically acceptable non-toxic acids or bases including inorganic acids and bases and organic acids and bases. When the compounds of the present invention are basic, salts may be prepared from pharmaceutically acceptable non-toxic acids including inorganic and organic acids. Suitable pharmaceutically acceptable acid addition salts for the compounds of the present invention include acetic, adipic, alginic, ascorbic, aspartic, benzenesulfonic (besylate), benzoic, boric, butyric, camphoric, camphorsulfonic, carbonic, citric, ethanedisulfonic, ethanesulfonic, ethylenediaminetetraacetic, formic, fumaric, glucoheptonic, gluconic, glutamic, hydrobromic, hydrochloric, hydroiodic, hydroxynaphthoic, isethionic, lactic, lactobionic, laurylsulfonic, maleic, malic, mandelic, methanesulfonic, mucic, naphthylenesulfonic, nitric, oleic, pamoic, pantothenic, phosphoric, pivalic, polygalacturonic, salicylic, stearic, succinic, sulfuric, tannic, tartaric acid, teoclatic, p-toluenesulfonic, and the like. When the compounds contain an acidic side chain, suitable pharmaceutically acceptable base addition salts for the compounds of the present invention include, but are not limited to, metallic salts made from aluminum, calcium, lithium, magnesium, potassium, sodium and zinc or organic salts made from lysine, arginine, N,N′-dibenzylethylenediamine, chloroprocaine, choline, diethanolamine, ethylenediamine, meglumine (N-methylglucamine) and procaine. Further pharmaceutically acceptable salts include, when appropriate, nontoxic ammonium cations and carboxylate, sulfonate and phosphonate anions attached to alkyl having from 1 to 20 carbon atoms.

Also provided herein is a pharmaceutical composition comprising a compound disclosed above, or a pharmaceutically acceptable salt form thereof, and a pharmaceutically acceptable carrier, diluent, or excipient.

While it may be possible for the compounds disclosed herein to be administered as the raw chemical, it is preferable to present them as a pharmaceutical composition. According to a further aspect, the present invention provides a pharmaceutical composition comprising a compound of formula I or a pharmaceutically acceptable salt thereof, together with one or more pharmaceutically carriers thereof and optionally one or more other therapeutic ingredients. The carrier(s) must be “acceptable” in the sense of being compatible with the other ingredients of the formulation and not deleterious to the recipient thereof. In one embodiment, the pharmaceutically acceptable carrier is selected from the group consisting of a liquid filler, a solid filler, a diluent, an excipient, a solvent, and an encapsulating material.

Pharmaceutically acceptable carriers (e.g., additives such as diluents, immunostimulants, adjuvants, antioxidants, preservatives and solubilizing agents) are nontoxic to the cell or subject being exposed thereto at the dosages and concentrations employed. Examples of pharmaceutically acceptable carriers include water, e.g., buffered with phosphate, citrate and another organic acid. Representative examples of pharmaceutically acceptable excipients that may be useful in the present disclosure include antioxidants such as ascorbic acid; low molecular weight (less than about 10 residues) polypeptides; proteins, such as serum albumin, gelatin, or immunoglobulins; adjuvants (selected so as to avoid adjuvant-induced toxicity, such as a (3-glucan as described in U.S. Pat. No. 6,355,625, which is hereby incorporated by reference in its entirety, or a granulocyte colony stimulating factor (GCSF)); hydrophilic polymers such as polyvinylpyrrolidone; amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt forming counterions such as sodium; and/or nonionic surfactants such as TWEEN®, polyethylene glycol (PEG), and PLURONICS®.

In one embodiment, the composition may further comprise an adjuvant. Suitable adjuvants are known in the art and include, without limitation, flagellin, Freund's complete or incomplete adjuvant, aluminum hydroxide, lysolecithin, pluronic polyols, polyanions, peptides, oil emulsion, dinitrophenol, iscomatrix, and liposome polycation DNA particles.

The formulations include those suitable for parenteral (including subcutaneous, intradermal, intramuscular, intravenous and intraarticular), rectal and topical (including dermal, buccal, sublingual and intraocular) administration. The most suitable route may depend upon the condition and disorder of the recipient. The formulations may conveniently be presented in unit dosage form and may be prepared by any of the methods well known in the art of pharmacy. All methods include the step of bringing into association a compound disclosed herein or a pharmaceutically acceptable salt thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations for parenteral administration include aqueous and non-aqueous sterile injection solutions which may contain anti-oxidants, buffers, bacteriostats and solutes which render the formulation isotonic with the blood of the intended recipient. Formulations for parenteral administration also include aqueous and non-aqueous sterile suspensions, which may include suspending agents and thickening agents. The formulations may be presented in unit-dose of multi-dose containers, for example sealed ampoules and vials, and may be stored in a freeze-dried (lyophilized) condition requiring only the addition of a sterile liquid carrier, for example saline, phosphate-buffered saline (PBS) or the like, immediately prior to use. Extemporaneous injection solutions and suspensions may be prepared from sterile powders, granules and tablets of the kind previously described.

The term “package insert” is used to refer to instructions customarily included in commercial packages of therapeutic products that contain information about the indication(s), usage, dosage, administration, contraindications, and/or warnings concerning the use of such therapeutic products.

It will be recognized that the compounds of this invention can exist in radiolabeled form, i.e., the compounds may contain one or more atoms containing an atomic mass or mass number different from the atomic mass or mass number usually found in nature. Radioisotopes of hydrogen, carbon, phosphorous, fluorine, and chlorine include ²H, ³H, ¹³C, ¹⁴C, ¹⁵N ³⁵S¹⁸F, and ³⁶Cl, respectively. Compounds that contain those radioisotopes and/or other radioisotopes of other atoms are within the scope of this invention. Tritiated, i.e. ³H, and carbon-14, i.e., ¹⁴C, radioisotopes are particularly preferred for their ease in preparation and detectability. Compounds that contain isotopes ¹¹C, ¹³N, ¹⁵O and ¹⁸F are well suited for positron emission tomography. Radiolabeled compounds of formula I of this invention and prodrugs thereof can generally be prepared by methods well known to those skilled in the art. Conveniently, such radiolabeled compounds can be prepared by carrying out the procedures disclosed in the Examples and Schemes by substituting a readily available radiolabeled reagent for a non-radiolabeled reagent.

Preparation of compounds can involve the protection and deprotection of various chemical groups. The need for protection and deprotection, and the selection of appropriate protecting groups, can be readily determined by one skilled in the art. Suitable groups for that purpose are discussed in standard textbooks in the field of chemistry, such as Protective Groups in Organic Synthesis by T. W. Greene and P. G. M. Wuts [John Wiley & Sons, New York, 1999], in Protecting Group Chemistry, 1^{st} Ed., Oxford University Press, 2000; and in March's Advanced Organic chemistry: Reactions, Mechanisms, and Structure, 5^{th} Ed., Wiley-Interscience Publication, 2001.

### EXAMPLES

### Example 1. General Procedure for Conjugates of Permutation A (See FIG. 1)

1 mg of Trastuzumab (SydLab) in 0.5 ml of PBS was treated with 4 μl of 0.5 mg/ml PNGase F (and incubated at 37° C. for 48 hours to achieve complete deglycosylation. After complete deglycosylation was assessed using LCMS, 240 μl of 0.1 Sorenson's phosphate buffer (pH 6) was added followed by 20 μl of 30 mM of 2, 2′-disulfanediydiethanamine dichloride and 70 mg of transglutaminase powder (Ajimoto Activa). Note that extensive vortexing was needed to create a homogeneous solution. The mixture was vortexed vigorously and incubated at 37° C. for 24-72 h (typically 48 h). The antibody solution was transferred into a protein A column and excess reagents were washed off with PBS. The resin-bound antibody was treated with PBS 93.3 μL of PBS and 6.67 μl (10 eq) of 10 mM Sodium Diphenylphosphinobenzene-3-sulfonate (TPPMS) and incubated at room temperature for 2 hours. (Just enough volume to cover the resin) The excess reagents were removed by centrifugation at 100 g for 1 min. A linker payload solution was prepared consisting of 11 μl (8 eq) of mcValCitPABC_A6 in DMA, 93 μl of DMA and 546 μl of PBS. This solution was added to the resin and the mixture was vortexed and allowed to stand at rt overnight. After washing twice with PBS, the mono-loaded ADCs were eluted from the protein A column by adding 400 μl of glycine buffer (pH 2.7), while 30 μl of Tris buffer (pH 9.0) was added to the collecting tube. Eluate was collected by centrifugation at 100 g for 1 min. This elution was conducted thrice to collect mono-loaded ADC. The material was buffer exchanged into PBS using a 30K Pierce Concentrator (Thermofisher, Ref:885020). Final ADCs were diluted to 1 mL in PBS and the drug-antibody ratio was assessed via LCMS and ADC recovery was assessed using the Nanodrop.

Prior to initiating the hinge conjugation step, 50 μl of 0.5M EDTA stock solution was added to the ADCs (in PBS) followed by 12 equivalents of 5 mM Tris(2-carboxyethyl)phosphine hydrochloride(TCEP). After incubation for 2 h, the excess TCEP was removed by buffer exchange using a 30K Pierce concentrator, washing twice with 5 mM EDTA in PBS. The reduced ADCs were collected in 1 ml of 5 mM EDTA in PBS and treated with 7 μl (10 eq) of 10 mM of mc_MMAF. After incubating at room temperature for 2 hour, the resultant dual ADC solution was buffer exchanged into PBS (30 kd spin column). Successful loading and drug-antibody ratio was assessed using LCMS. ADC percent recovery (i.e., protein concentration) was assessed using nanodrop. The amount of aggregation was assessed by SEC using BIORAD NGC Chromatography system or Agilent 1260 HPLC using a Tosoh TSKgel G3000 column. Dual ADC solutions were sterile-filtered using 0.22 μm PVDF filter (Fisher Scientific, Cat no. 09-720-3), labelled and stored at 4° C.

As discussed supra, there is a maximum of two Q295 sites on which Payload 1 may theoretically be loaded, for example, as illustrated in FIG. 1. However, some additional extraneous loading at other heavy chain sites (i.e., non-Q295 sites) may result in a measured DAR value that is greater than the maximum claimed value for d1 of “about 2.”

ADCs prepared using this method:
ADC389: Anti-Trop2-(Q295-mcValCitPABC-A6)-(mcMMAF)
ADC390: Anti-Her2-(Q295-mcValCitPABC-A6)-(mcMMAF)
ADC496: Anti-Trop2-(Q295-mcA6)-(mcMMAF)
ADC497: Anti-Her2-(Q295-mcA6)-(mcMMAF)

| | | | | Measured | Measured | | ΔMW of | ΔMW of | MW of | MW of |
|---|---|---|---|---|---|---|---|---|---|---|
| | | Pay- | Pay- | DAR | DAR | | major | major | major | major |
| | | load 1 | load 2 | (Pay- | (Pay- | % | HC | LC | HC | LC |
| ADC# | Antibody | (Q295) | (Hinge) | load 1) | load 2) | Agg. | peak | peak | peak | peak |
| ADC389 | Trastuzumab- | mcValCitPABC_A6 | mc_MMAF | 2 | 7.5 | 2.3 | 2814 | 929 | 52979 | 24371 |
| ADC390 | Sacituzumab | mcValCitPABC_A6 | mc_MMAF | 2 | 8.0 | 2.8 | 2818 | 929 | 52981 | 24266 |
| ADC496 | Trastuzumab- | mc_A6 | mc_MMAF | 2 | 8.0 | 1.9 | 2778 | 923 | 52530 | 24361 |
| ADC497 | Sacituzumab | mc_A6 | mc_MMAF | 2.8 | 8.0 | 2.2 | 2782 | 927 | 52674 | 24259 |

### Example 2. General Procedure For Conjugates Of PERMUTATION B (See FIG. 1)

1 mg of Trastuzumab (SydLab) in 0.5 ml of PBS was treated with 4 μl of 0.5 mg/ml PNGase F (and incubated at 37° C. for 48 hours to achieve complete deglycosylation. After complete deglycosylation was assessed using LCMS, 240 μl of 0.1 Sorenson's phosphate buffer (pH 6) was added followed by 20 μl of 30 mM of 2, 2′-disulfanediydiethanamine dichloride and 70 mg of transglutaminase powder (Ajimoto Activa). Note that extensive vortexing was needed to create a homogeneous solution. The mixture was vortexed vigorously and incubated at 37° C. for 24-72 h (typically 48 h). The antibody solution was transferred into a protein A column and excess reagents were washed off with PBS. The resin-bound antibody was treated with PBS 93.3 μL of PBS and 6.67 μl (10 eq) of 10 mM Sodium Diphenylphosphinobenzene-3-sulfonate (TPPMS) and incubated at room temperature for 2 hours. (Just enough volume to cover the resin) The excess reagents were removed by centrifugation at 100 g for 1 min. A linker payload solution was prepared consisting of 8 μl (8 eq) of mcMMAF in DMA and 92 μL of PBS. This solution was added to the resin and the mixture was vortexed and allowed to stand at rt overnight. After washing twice with PBS, the mono-loaded ADCs were eluted from the protein A column by adding 400 μl of glycine buffer (pH 2.7), while 30 μl of Tris buffer (pH 9.0) was added to the collecting tube. Eluate was collected by centrifugation at 100 g for 1 min. This elution was conducted thrice to collect mono-loaded ADC. The material was buffer exchanged into PBS using a 30K Pierce Concentrator (Thermofisher, Ref.885020). Final ADCs were diluted to 1 mL in PBS and the drug-antibody ratio was assessed via LCMS and ADC recovery was assessed using the Nanodrop.

Prior to initiating the hinge conjugation step, 50 μl of 0.5M EDTA stock solution was added to the ADCs (in PBS) followed by 12 equivalents of 5 mM Tris(2-carboxyethyl)phosphine hydrochloride (TCEP). After incubation for 2 h, the excess TCEP was removed by buffer exchange using a 30K Pierce concentrator, washing twice with 5 mM EDTA in PBS. The reduced ADCs were collected in 1 ml of 5 mM EDTA in PBS and treated with a solution consisting of 26 μL of 5 mM mcValCitPABC_A6, 254 μL of DMA, and 1020 μL of PBS containing 5 mM EDTA. After incubating at room temperature for 2 hour and 4° C. for 18 h, the resultant dual ADC solution was buffer exchanged into PBS (30 kd spin column). Successful loading and drug-antibody ratio was assessed using LCMS. ADC percent recovery (i.e., protein concentration) was assessed using nanodrop. The amount of aggregation was assessed by SEC using BIORAD NGC Chromatography system or Agilent 1260 HPLC using a Tosoh TSKgel G3000 column. Dual ADC solutions were sterile-filtered using 0.22 μm PVDF filter (Fisher Scientific, Cat no. 09-720-3), labelled and stored at 4° C.

The following ADCs were prepared using this method:
ADC387: Anti-Trop2-(Q295-mcMMAF)-(mcValCitPABC-A6)
ADC388: Anti-Her2-(Q295-mcMMAF)-(mcValCitPABC-A6)
ADC494: Anti-Trop2-(Q295-mcMMAF)-(mc_A6)
ADC495: Anti-Her2-(Q295-mcMMAF)-(mc_A6)
ADC491: Anti-Trop2-(Q295-mcMMAE)-(mc_A6)

| | | | | Measured | Measured | | ΔMW of | ΔMW of | MW of | MW of |
|---|---|---|---|---|---|---|---|---|---|---|
| | | Pay- | Pay- | DAR | DAR | | major | major | major | major |
| | | load 1 | load 2 | (Pay- | (Pay- | % | HC | LC | HC | LC |
| ADC# | Antibody | (Q295) | (Hinge) | load 1) | load 2) | Agg. | peak | peak | peak | peak |
| ADC387 | Trastuzumab | mc_MMAF | mcValCitPABC_A6 | 2 | 7.5 | 23 | 2839 | 948 | 52979 | 24387 |
| ADC388 | Sacituzumab | mc_MMAF | mcValCitPABC_A6 | 2 | 8.0 | 7.9 | 2836 | 951 | 52979 | 24282 |
| ADC494 | Trastuzumab | mc_MMAF | mc_A6 | 1.9 | 8.4 | 1.2 | 1618 | 537 | 51754 | 23975 |
| ADC495 | Sacituzumab | mc_MMAF | mc_A6 | 2.2 | 8.3 | 1.4 | 1634 | 540 | 51897 | 23873 |
| ADC491 | Sacituzumab | mcValCitPABC_MMAE | mc_A6 | 2.0 | 4.8 | 2.9 | 543 | 539 | 51215 | 23874 |

### Example 3: Preparation of Dual-Payload mcMMAF+mcAsnAsnA6 and mcMMAF+mcA6 ADCs

Step 1: Deglycosylation. 20 mg of Trastuzumab or Sacituzumab (SydLab) in 2 ml of DPBS was treated with 40μl of 0.5 mg/ml PNGase F and incubated at 37° C. for 48 hrs to achieve deglycosylation. Successful deglycosylation was assessed by LCMS.

Step 2: Cystamine conjugation. The deglycosylated mAb (15 mg) was diluted to 2 mg/mL (7.5 mL) in PBS and treated with 4.5 mL of Sorenson's phosphate buffer (pH 6) followed by 300 μL of 30 mM of Cystamine and 600 mg of transglutaminase powder (Ajinimoto Activa). Note that extensive vortexing was needed to create a turbid homogeneous solution. After incubation at 37° C. for 48 h, the cystamine conjugation was assessed using LCMS. The product was purified using protein A column to remove transglutaminase and PNGase F. In short, a Protein A HP SpinTrap (Fisher, Cat No: 45-001-483) was washed with PBS. The crude antibody mixture was loaded onto the column. After gentle rotation, the unbound proteins and small molecules were then removed by centrifugation for 0.5 min at 0.1 rcf. The washing was repeated twice (500 μL each). The conjugate was eluted with 400 μL of 0.2M glycine (pH 2.7) into a tube preloaded with 30 μL of 1 M tris, pH 9. This elution was performed a second time and both eluents were pooled. The product was buffer exchanged into DPBS by centrifugal filtration (Pierce Protein Concentrator PES, 30K MWCO).

Step 3: Q295 conjugation of MMAF. Prior to initiating the Q295 conjugation step, a stock solution of 5 mM EDTA in PBS was added to the cystamine conjugated antibody (10 mg) in DPBS to give a concentration of 4 mg/mL. Sodium Diphenylphosphinobenzene-3-sulfonate (TPPMS) (33.33 μL of 10 mM stock, 5 eq) was added and the mixture was incubated at room temperature for 2 hrs. DMA was added to give a 5% (v/v, final) mixture followed by the addition of 12 eq of 10 mM of mcMMAF stock solution in DMA (93.3 μl, 7 eq). This reaction mixture (2.5 mL final reaction volume) was then vortexed and allowed to stand at rt for another 1 hr. The DAR was assessed using LCMS. The crude material was buffer exchanged to DPBS by centrifugal filtration (Pierce Protein Concentrator PES, 30K MWCO). Protein concentration was determined by UV absorption, DAR was determined by LCMS, and aggregation was assessed by SEC. ADCs were sterile-filtered using 0.22 μm PVDF filter and stored at 4° C.

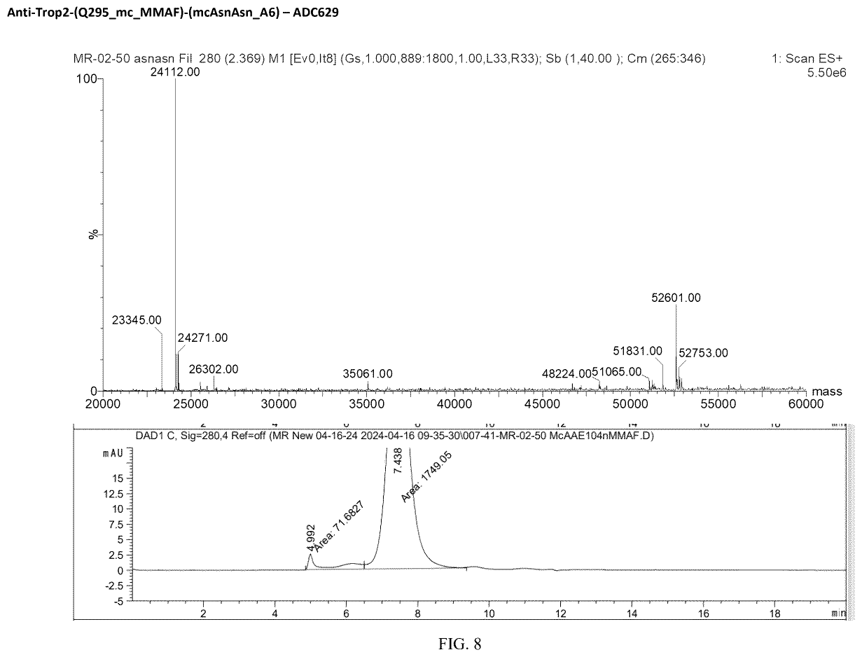

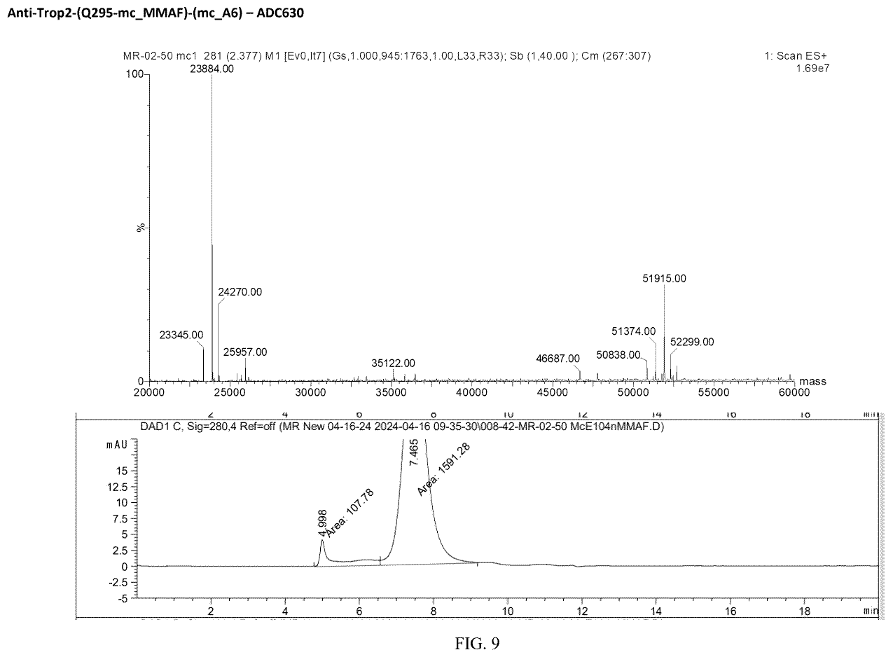

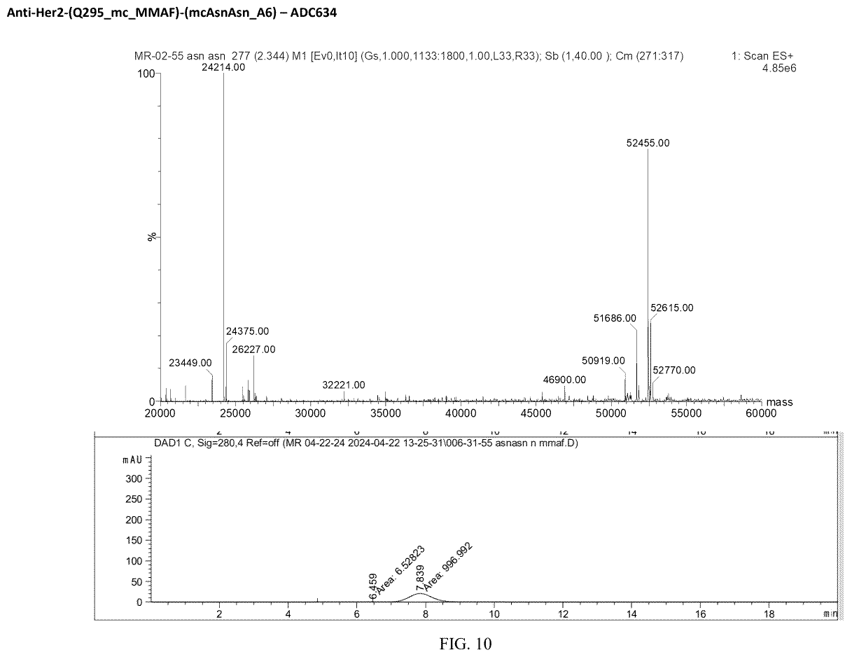

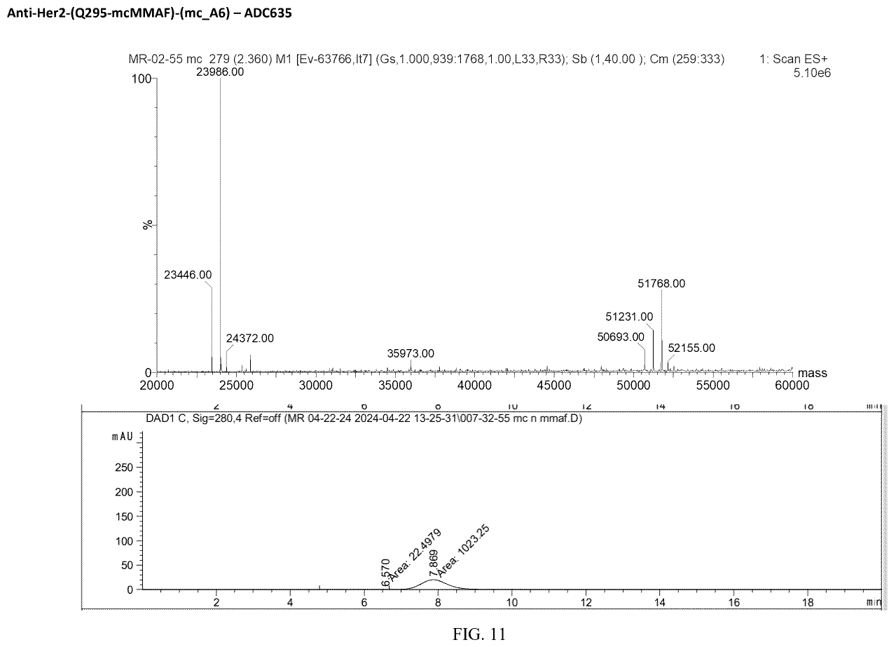

Step 4: Cysteine conjugation of mcAsnAsnA6 or mcA6. The mono-loaded Q295 ADC (3 mg, from step 3) were treated with 5 mM EDTA stock solution to give a 2 mg/mL solution. Tris(2-carboxyethyl)phosphine hydrochloride(TCEP) (24 μL of 5 mM, 6 eq) was added and the sample was incubated at 37° C. for 2 hrs. DMA was added to give 10% organic (final) followed by the 12 eq (48 μL) of 10 mM of the appropriate linker payload (1.5 mL total rxn volume). After incubation at room temperature for 2 hr, the crude product was buffer exchanged by ultrafiltration (Pierce Protein Concentrator PES, 30K MWCO) to DPBS. Protein concentration was determined by UV absorption, DAR was determined by LCMS, and aggregation was assessed by SEC. ADCs were sterile-filtered using 0.22 μm PVDF filter and stored at 4° C. Results are shown in FIGS. 8-11. Single-payload ADCs also were made to confirm that the dual-payload ADCs retain the activity of the single-payload ADCs.

The following dual-payload ADCs were prepared using this method
Trop2-Saci-_(mc_MMAF)-(McAsnAsn A6)
Trop2-Saci-_(mc_MMAF)-(mc_A6)

| | | | | Measured | Measured | | ΔMW of | ΔMW of | MW of | MW of |
|---|---|---|---|---|---|---|---|---|---|---|
| | | LP 1 | LP 2 | DAR | DAR | | Major | Major | Major | Major |
| ADC# | Antibody | (Q295) | (Cysteine) | (LP1) | (LP2) | % Agg. | HC peak | LC peak | HC peak | LC peak |
| ADC629 | Anti-Trop2 | mc_MMAF | McAsnAsn_A6 | 2.7 | 6.9 | 3.9 | 3232 | 768 | 52601 | 24112 |
| ADC630 | Anti-Trop2 | mc_MMAF | mc_A6 | 2.7 | 6.9 | 6.3 | 2546 | 540 | 51915 | 23884 |

ΔMWs for ADCs are calculated from cystamine conjugated Antibody.

| | | | | Measured | Measured | | ΔMW of | ΔMW of | MW of | MW of |
|---|---|---|---|---|---|---|---|---|---|---|
| | | Payload 1 | Payload 2 | DAR | DAR | | Major | Major | Major | Major |
| ADC# | Antibody | (Q295) | (Hinge) | (Payload 1) | (Payload 2) | % Agg. | HC peak | LC peak | HC peak | LC peak |
| ADC634 | Anti-HER2 | mc_MMAF | McAsnAsn_A6 | 2.4 | 7.1 | 0.6 | 3224 | 763 | 52455 | 24214 |
| ADC635 | Anti-HER2 | mc_MMAF | mc_A6 | 2.4 | 6.5 | 2 | 2537 | 535 | 51768 | 23986 |

ΔMWs for ADCs are calculated from cystamine conjugated Antibody.

### Example 4. In Vitro Cytotoxicity Assays

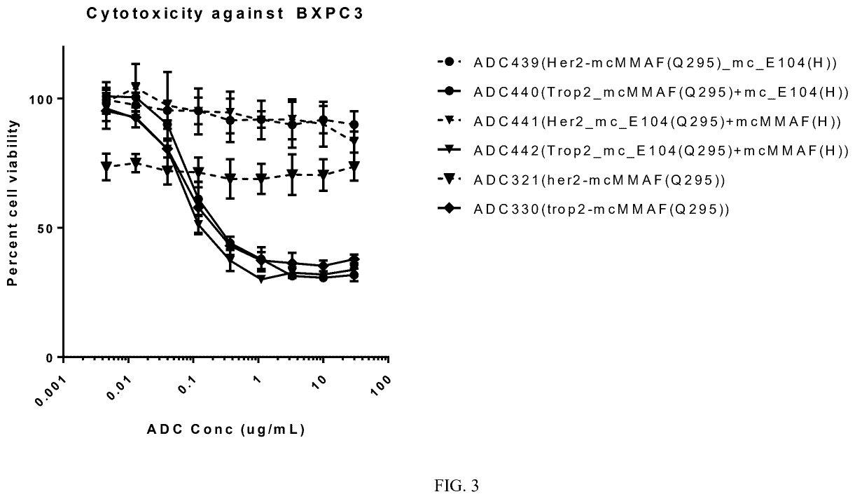

In vitro cytotoxicity studies of Dual ADCs and Mono-loaded ADCs were performed on the trop2-expressing pancreatic adenocarcinoma BxPC3 cell line. Cells were seeded on black walled clear bottom 96-well plates at 25,000 cells per well. Cells were treated with 3-fold serially diluted ADCs in triplicates. The negative control was DPBS treated wells. Cell viability was determined by CellTiter-Glo Luminescent Cell Viability Assay (Promega, Madison, WI) 72 hours after treatment. Luminescence was measured using Thermo Scientific™ Varioskan™ LUX. IC50 values were calculated by GraphPad Prism 7 software. Results are shown in FIG. 2 and FIG. 3.

### Example 5. In Vitro Immune Modulation Assay

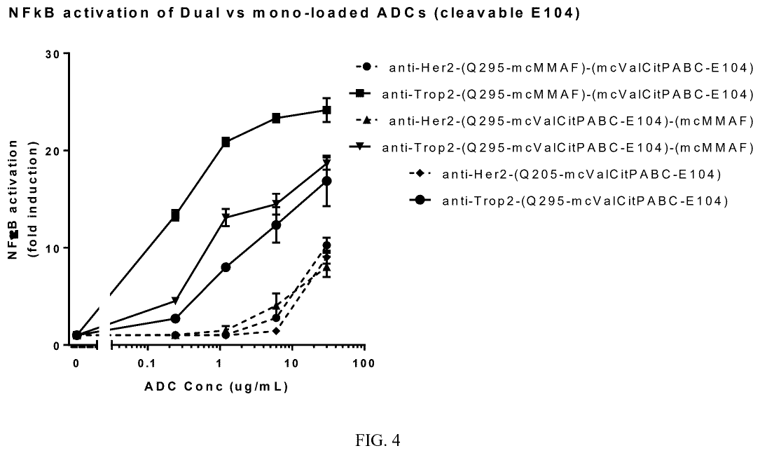

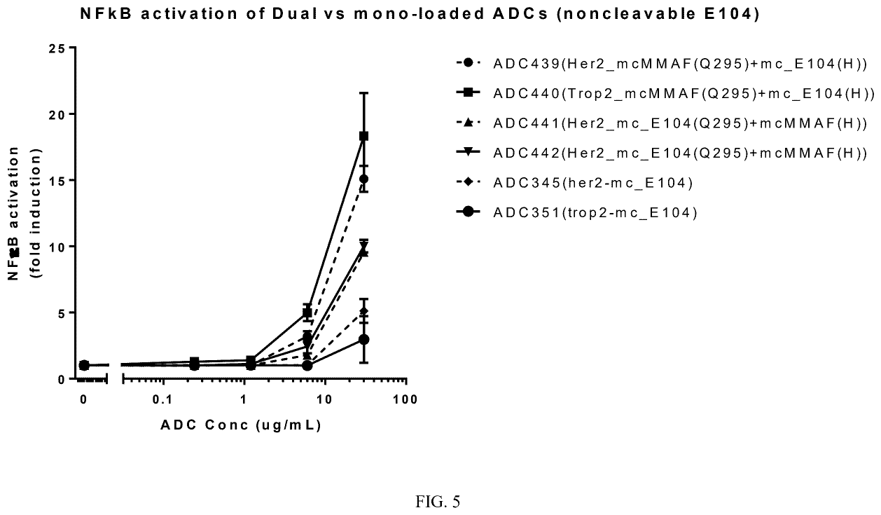

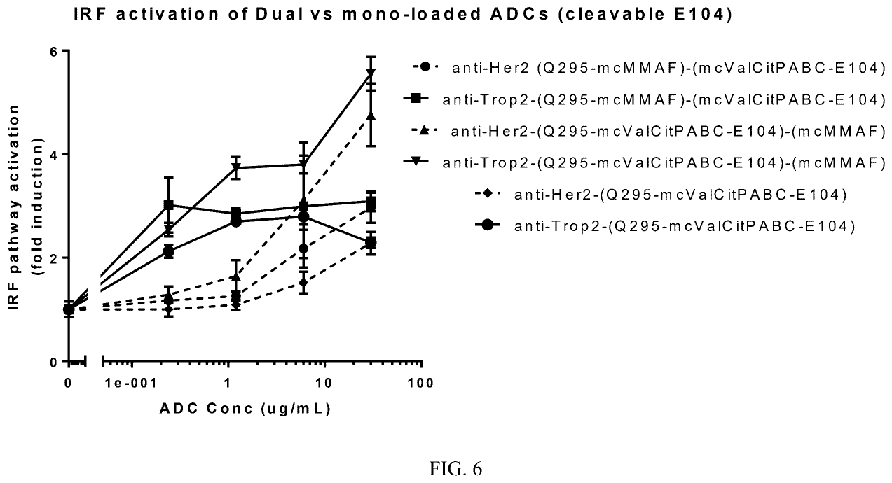

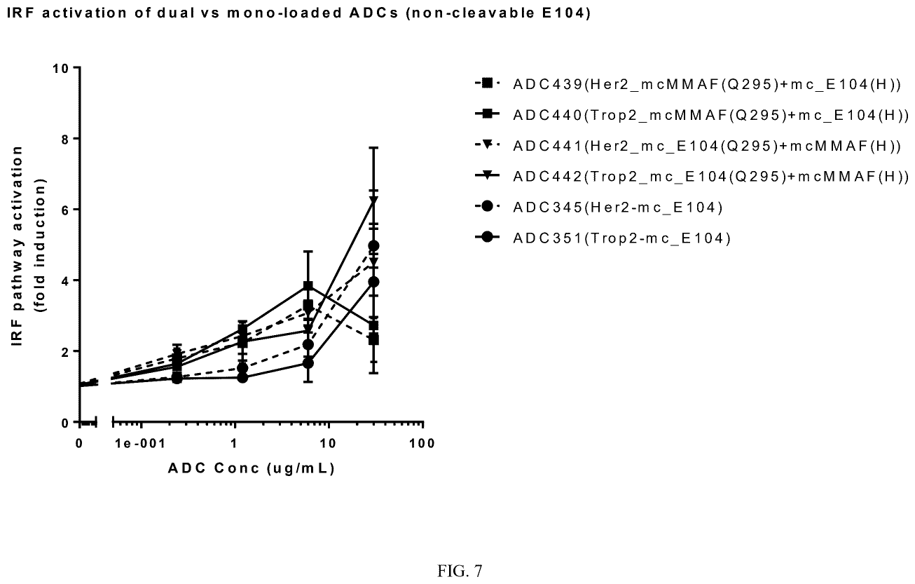

In vitro immune modulation studies of Dual ADCs in comparison to mono-loaded ADCs were done using a co-culture of trop2-expressing pancreatic adenocarcinoma BxPC3 cell line and RAW Dual cells. RAS Dual cells have a dual-reporter system in which the NFκB pathway activation can be assessed by SEAP production (QB assay) and the IRF7 pathway activation can be assessed by luciferase expression (QL assay). Cells were seeded in 96-well plates consisting of 25,000 BXPC-3 cell and 20,000 RAW Dual cells. Cells were treated with 5-fold serially diluted ADCs in triplicates. Controls include cells treated with DPBS, A6(payload) and naked antibodies(Trop2 and Her2). Immune modulation was measured 51 hours after treatment using Quanti-blue(QB) kit (InvivoGen, cat:rep-qblb) by adding 20 μl of cell supernatant to 180 μl of QB solution and incubated for 24 hrs. Absorbance was measured using Thermo Scientific™ Varioskan™ LUX. Immune modulation was also assessed using QUANTI-luc kit (InvivoGen, cat: #reglc) and luminescence was measured using Thermo Scientific™ Varioskan™ LUX. Data were analyzed using GraphPad Prism 7 software. Results are shown in FIGS. 4-7.

While several aspects of the present invention have been described and depicted herein, alternative aspects may be effected by those skilled in the art to accomplish the same objectives. Accordingly, it is intended by the appended claims to cover all such alternative aspects as fall within the true spirit and scope of the invention.

1. An antibody-drug conjugate of Formula (A): 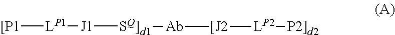
wherein:
P1 comprises a TLR7 agonist, MMAE, or MMAF;
L^{P1} comprises a cleavable or noncleavable linker;
J1 comprises a first conjugation handle;
S^{Q} is —S(C₁-C₆)alkylNH—, wherein the NH is attached to the carbonyl of a Q295 residue of Ab;
Ab comprises an antibody;
J2 comprises a second conjugation handle attached at a sulfur atom of a cysteine residue of Ab;
L^{P2} comprises a cleavable or noncleavable linker; and
P2 comprises a TLR7 agonist, MMAE or MMAF;
wherein:
d1 is between 1 and about 2;
d2 is between about 2 and about 10;
L^{P1} and L^{P2} may be the same or different;
J1 and J2 may be the same or different; and
when P1 is a TLR7 agonist, P2 is selected from MMAE or MMAF; and when P1 is MMAE or MMAF, P2 is a TLR7 agonist.

2. The antibody-drug conjugate of claim 1, wherein P1 or P2 is a TLR7 agonist selected from a compound of Formula (I) or Formula (II): 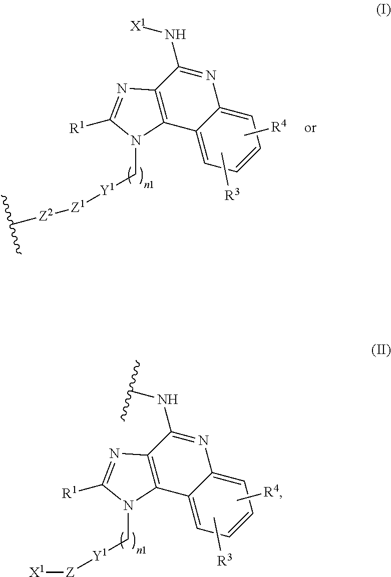
wherein:
R¹ is selected from C₁-C₁₀ alkyl, C₁-C₁₀ oxaalkyl, and C₁-C₁₀ azaalkyl;
R³ and R⁴ are each independently selected from hydrogen, C₁-C₅ alkyl, and C₁-C₅ alkoxy;
n1 is 1 or 2;
Y¹ is independently selected from optionally substituted aryl and optionally substituted heteroaryl;
Z¹ is selected from —NR^{Z}—, —O—, —NR^{Z}C(O)—, —NR^{Z}C(O)—O—, and —NR^{Z}SO₂—;
Z² is absent, or is selected from —(C₁-C₅)hydrocarbon, (C₁-C₅)hydrocarbon-NH—, —NH—(C₁-C₅)hydrocarbon, and a 5-to 8-membered nitrogen-containing heterocycle, wherein the nitrogen is attached to L^{P1} or L^{P2};
Z is independently selected from —NR^{Z}—, —NOR^{Z}C(O)—, and —O—;
R^{Z} is independently selected in each instance from hydrogen, C₁-C₈ hydrocarbon, C₁-C₈ oxaalkyl, C₁-C₈ azaalkyl, heteroaryl, C₁-C₄ arylalkyl, and a 5-to 8-membered heterocyclic ring;
X¹ is independently selected from R^{Z}, —C(O)—R^{Z}, —C(O)—O—R^{Z}, —C(O)—N—(R^{Z})₂, —(CH₂)ₖNR^{Z}C(O)—(C₁-C₆)alkyl, —(CH₂)ₖNR^{Z}C(O)—O—(C₁-C₄)alkyl, —SO₂—NH—R^{Z}, and —SO₂—R^{Z};
k is an integer from 1 to 8; and [symbol]
is the point of attachment to L^{P1} or L^{P2}.

3. The antibody-drug conjugate of claim 2, wherein P1 or P2 is of Formula (I), and:
R¹ is n-butyl;
R³ and R⁴ are each hydrogen;
n1 is 1;
Y¹ is phenyl or pyridyl, each of which is unsubstituted or substituted with one or more of halogen, C₁-C₄ alkyl, C₁-C₄ alkoxy, C₁-C₄ haloalkyl, or C₁-C₄ haloalkoxy;
X¹ is hydrogen; and
Z¹ is —N(R^{Z})— or —O—.

4. The antibody-drug conjugate of claim 1, wherein P1 or P2 is selected from: 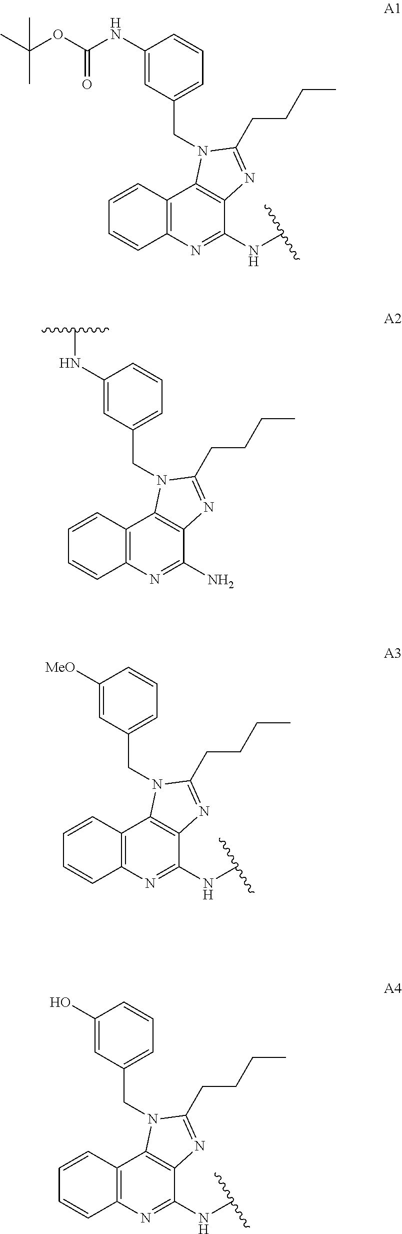 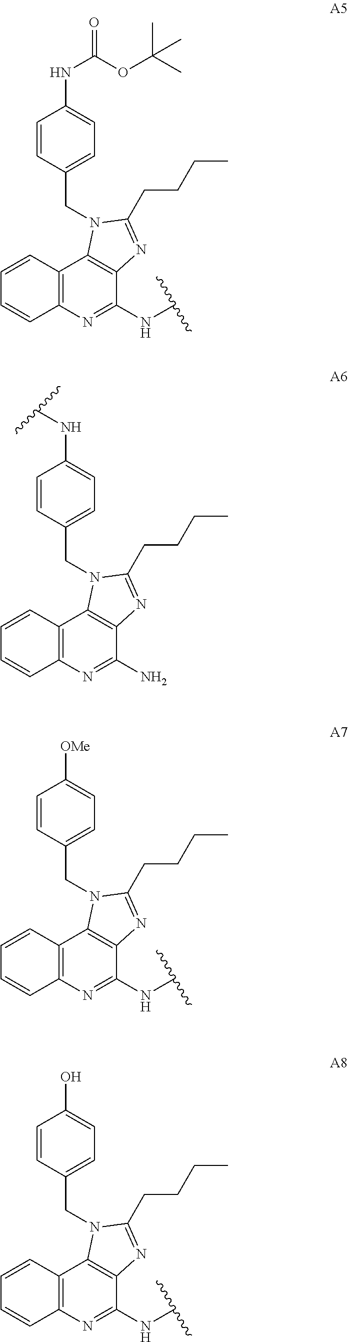 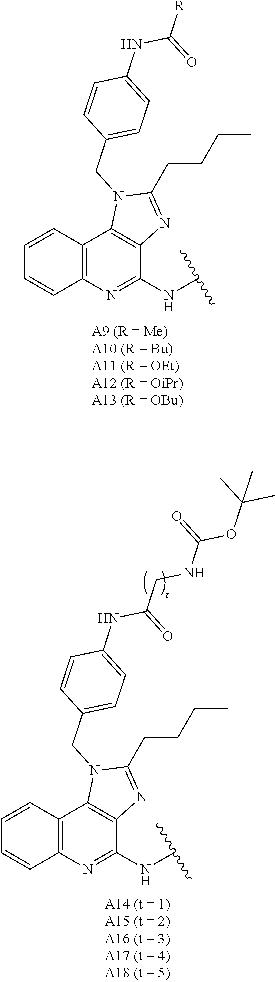 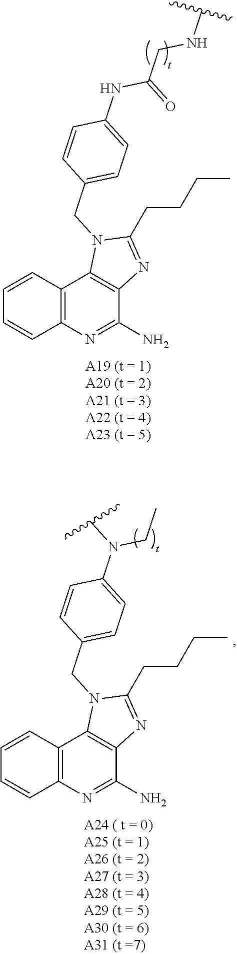
wherein [symbol] is the point of attachment to L^{P1} or L^{P2}.

5. The antibody-drug conjugate of claim 1,
wherein L^{P1} and L^{P2} are each independently selected from: 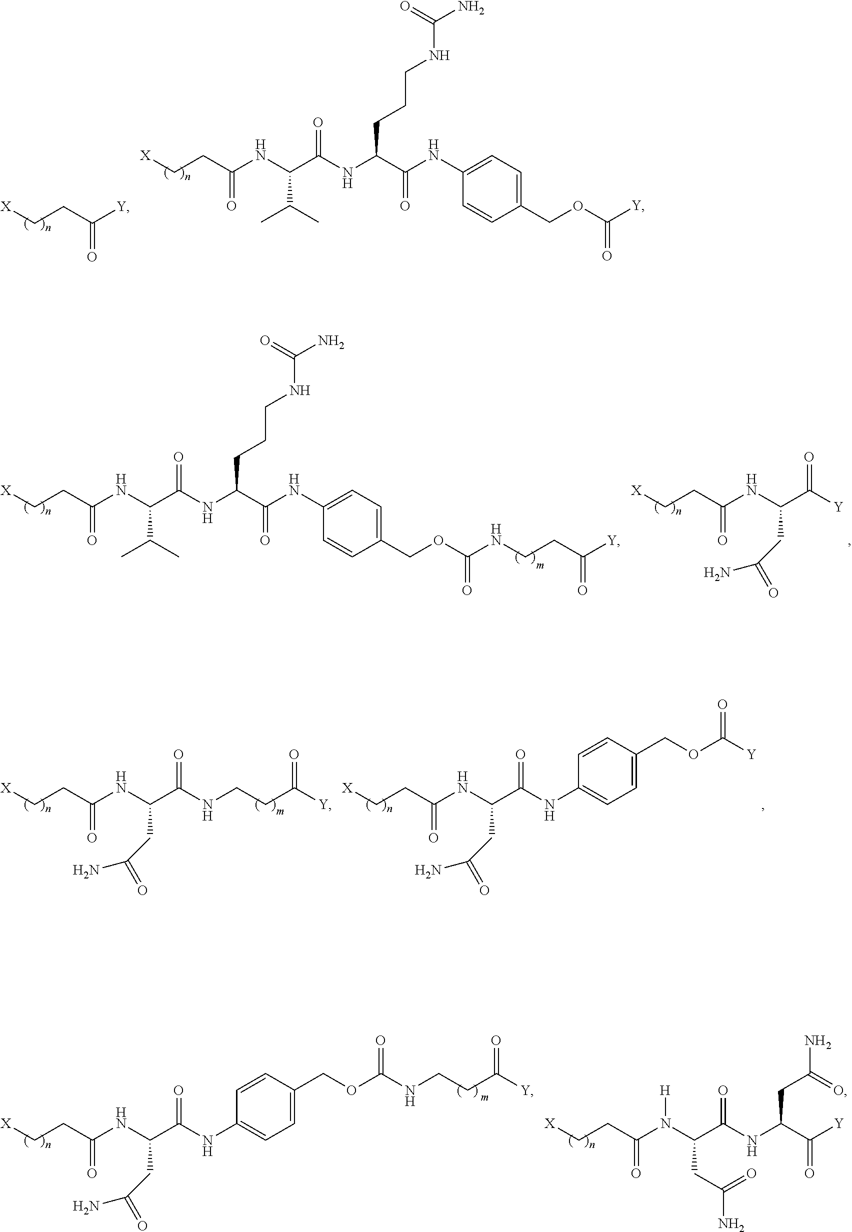 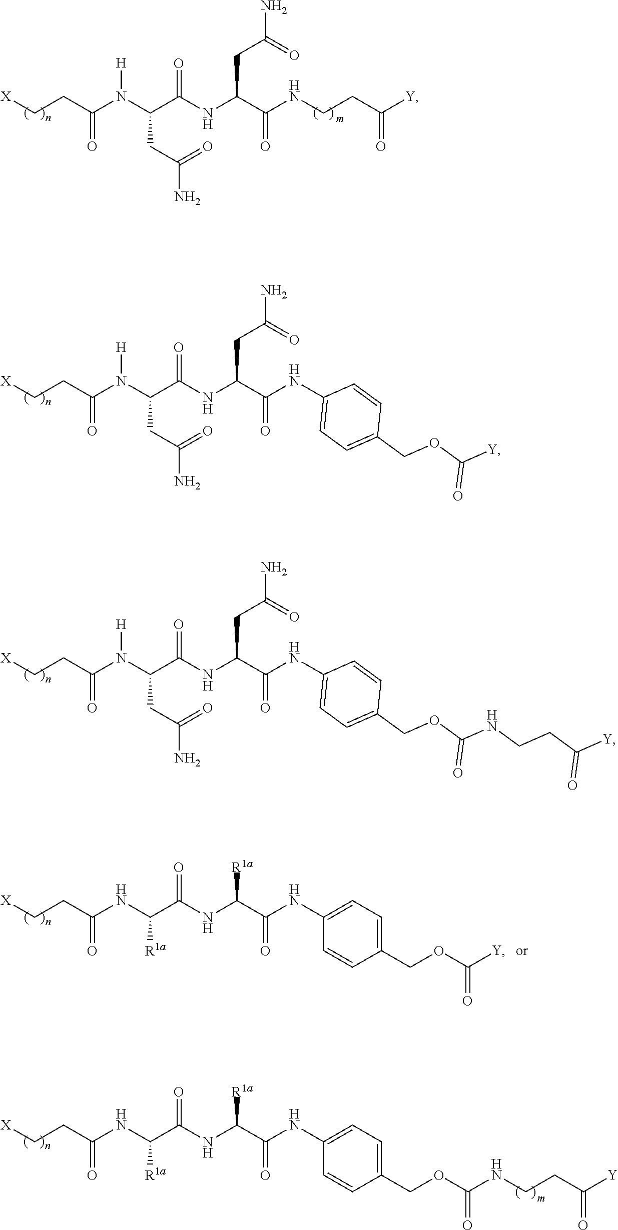
wherein:
R^{1a} is hydrogen, (C₁-C₄)alkyl, benzyl, CH₂OH, (CH₂)ᵥNH₂, CH₂CO₂H, CH₂CH₂CO₂H, CH₂CONH₂, or CH₂CH₂CONH₂;
v is 2, 3, 4, or 5;
m is 0, 1, 2, 3, 4, 5, 6, or 7;
n is 0, 1, 2, 3, 4, 5, 6, or 7;
Y is P1 or P2; and
X is J1 or J2.

6. The antibody-drug conjugate of claim 1, wherein J1 and J2 are each independently selected from: 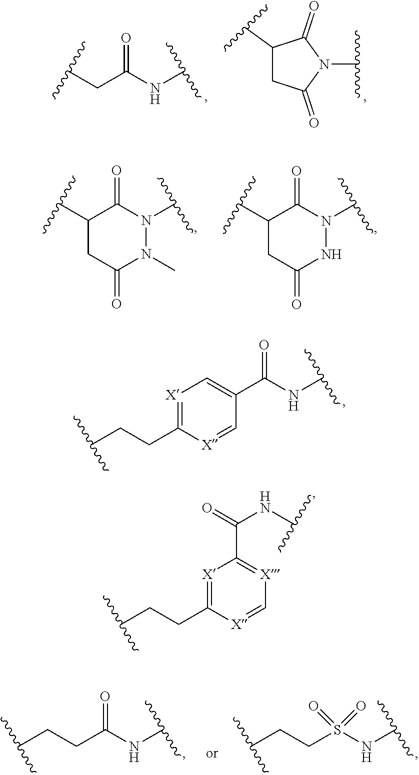
wherein X′, X″, and X′″ are each independently selected in each instance from CH or N; and
wherein the left-hand [symbol] is the point of attachment to S^{Q}, and the right-hand [symbol] is the point of attachment to L^{P1} or L^{P2}.

7. The antibody-drug conjugate according to claim 1, wherein:
L^{P1}-J1 or J2-L^{P2} is L1-L2-(L3)ₚ-(L4)_{q}-(L5)ᵣ;
L1 is a conjugation moiety;
L2 is a spacer unit selected from branched or unbranched C₁-C₁₂ alkyl, a PEG selected from PEG1 to PEG12, 1-12, 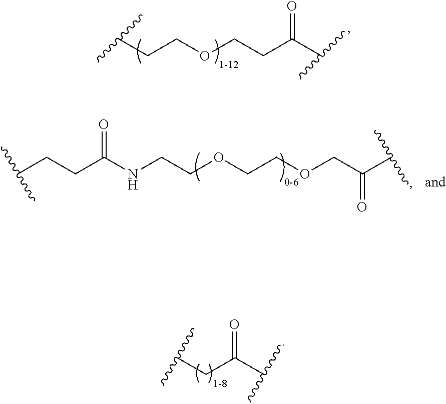
L3 is a peptide of 1 to 6 amino acids;
L4 is a self-immolative spacer;
L5 is carbonyl; and
p, q, and r are each independently selected from 0 and 1, wherein when p and q are each 0, r must be 0.

8. The antibody-drug conjugate of claim 7, wherein:
L1 is selected from; 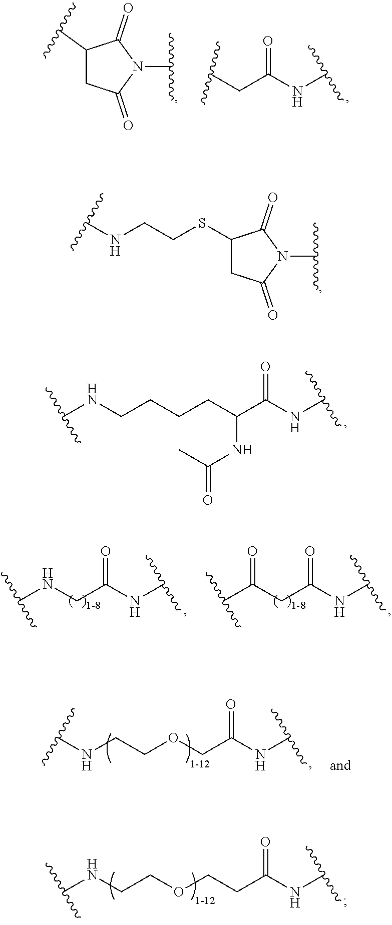
L2 is selected from 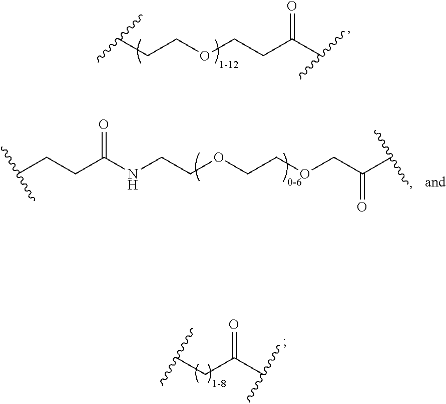
L3 is 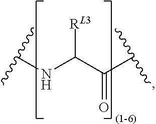
wherein R^{L3} is an amino acid side chain;
L4 is selected from: 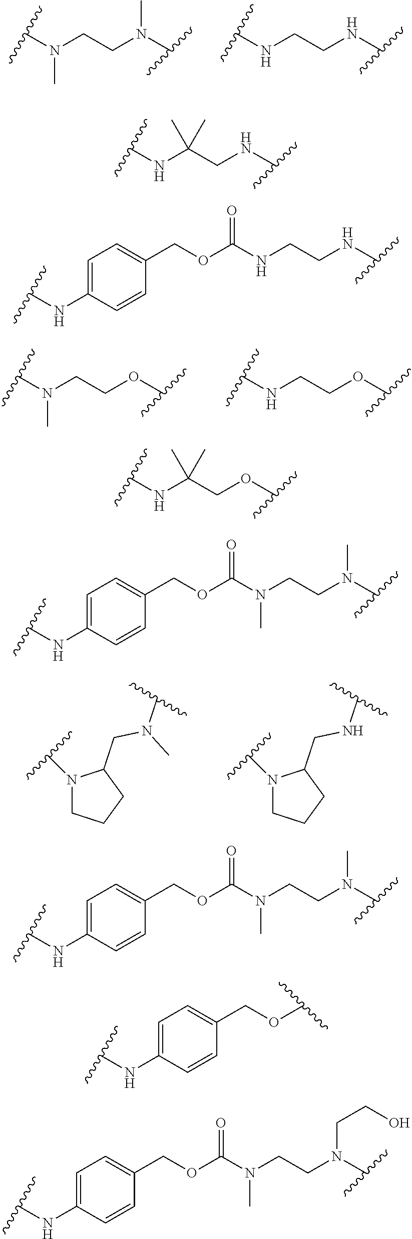 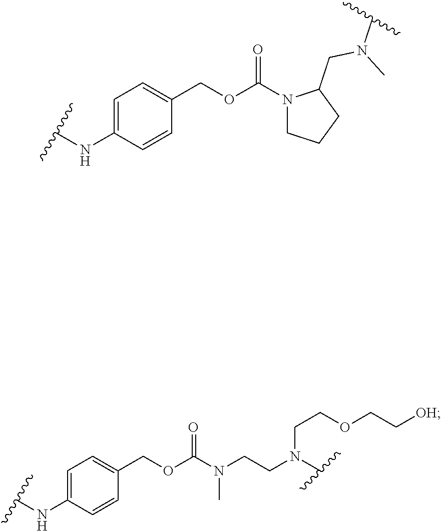 [SMILES: *Nc1ccc(COC(=O)N2CCCC2CN(*)C)cc1 ; *Nc1ccc(COC(=O)N(C)CCN(*)CCOCCO)cc1]
L5 is 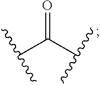 [SMILES: *C(*)=O]
and
p, q, and r are each independently 0 or 1, wherein when p and q are each 0, r must be 0.

9. The antibody-drug conjugate of claim 7, wherein L3 is selected from ValCit, GlyValCit, ValArg, PheLys, AlaAla, GlyGlyPheGly, AlaAlaAla, AlaAsn, AsnAsn, AsnAla, ValCitGlyPro, AsnGlyPro, AsnAsnGlyPro, Asn, GlyAsn, AsnAla, ProCitAla, ProAsnLeu, ProAsnAla, ProPheAla, ProPheGly, ProCitLeu, ProAsnPro, ProAsnSer, and ProAsnGly.

10. The antibody-drug conjugate of claim 7, wherein:
L1 is selected from 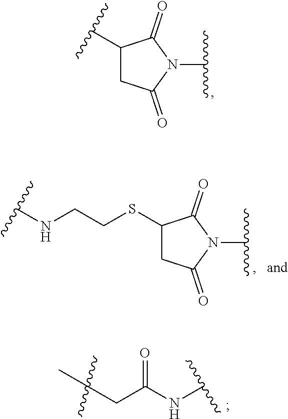 [SMILES: *C1CC(=O)N(*)C1=O ; *NCCSC1CC(=O)N(*)C1=O ; *CC(=O)N*]
L2 is 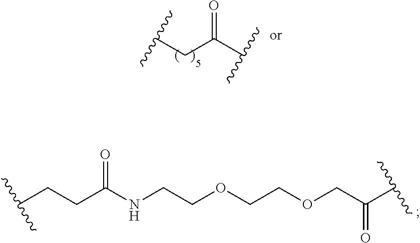
L3 is ValCit, GlyValCit, AsnAsn, Asn or AlaAla;
L4 is selected from: 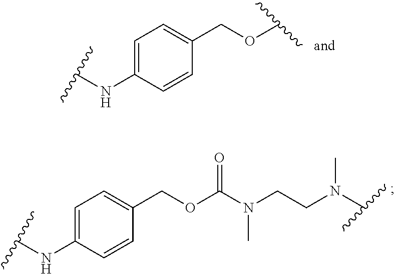 [SMILES: *Nc1ccc(CO*)cc1 ; *Nc1ccc(COC(=O)N(C)CCN(*)C)cc1]
L5 is 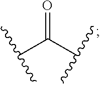 [SMILES: *C(*)=O]
and
p, q, and r are each 0 or p, q, and r are each 1.

11. The antibody-drug conjugate of claim 1, wherein S^{Q} is a C2 alkyl.

12. The antibody-drug conjugate of claim 1, wherein P1 is a TLR7 agonist and P2 is MMAE or MMAF.

13. The antibody-drug conjugate of claim 1, wherein P1 is MMAE or MMAF and P2 is a TLR7 agonist.

14. The antibody-drug conjugate of claim 1, wherein L^{P1}-J1 or J2-L^{P2} is selected from mc, mcValCitPABC, mcAsnAsn, mpAsnAsn, maAsnAsn, mcAsnAsnGlyPro, mcValCitGlyPro, mcAlaAlaGlyPro, mcAsnAsnPABC, or mcAlaAlaPABC.

15. (canceled)

16. The antibody-drug conjugate of claim 1, wherein L^{P1}-J1 or J2-L^{P2} is selected from mc, mcValCitPABC, mcAsnAsn, mpAsnAsn, maAsnAsn, mcAsnAsnGlyPro, mcValCitGlyPro, mcAlaAlaGlyPro, mcAsnAsnPABC, or mcAlaAlaPABC; and one of P1 or P2 is selected from a TLR agonist selected from structures A1 to A31, while the other of P1 or P2 is selected from MMAE or MMAF; and/or wherein one of P1 or P2 is selected from a TLR agonist selected from structures A6, A10, or A15 described above, while the other of P1 or P2 is selected from MMAE or MMAF.

17-19. (canceled)

20. The antibody-drug conjugate of claim 1, wherein P1-L^{P1}-J1 or J2-L^{P2}-P2 is selected from me-A6, me-A10, me-A15, me-MMAF, me-MMAE, mcValCitPABC-A6, mcValCitPABC-A10, mcValCitPABC-A15, mcAsnAsn-A6, mcAsnAsn-A10, mcAsnAsn-A15, mcValCitPABC-MMAF, mcValCitPABC-MMAE, mcAsnAsnGlyPro-A6, mcAsnAsnGlyPro-A10, mcAsnAsnGlyPro-A15, mcAsnAsnGlyPro-MMAF, mcAsnAsnGlyPro-MMAE, mcValCitGlyPro-A6, mcValCitGlyPro-A10, mcValCitGlyPro-A15, mpAsnAsn-A6, maAsnAsn-A6, mcValCitGlyPro-MMAF, mcValCitGlyPro-MMAE, mcAlaAlaGlyPro-A6, mcAlaAlaGlyPro-A10, mcAlaAlaGlyPro-A15, mcAlaAlaGlyPro-MMAF, mcAlaAlaGlyPro-MMAE, mcAsnAsnPABC-A6, mcAsnAsnPABC-A10, mcAsnAsnPABC-A15, mcAsnAsnPABC-MMAF, mcAsnAsnPABC-MMAE, mcAlaAlaPABC-A6, mcAlaAlaPABC-A10, mcAlaAlaPABC-A15, mcAlaAlaPABC-MMAF, or mcAlaAlaPABC-MMAE.

21. A pharmaceutical composition comprising the antibody-drug conjugate of claim 1 and a pharmaceutically acceptable carrier, diluent, or excipient.

22. A method for treating a tumor or abnormal cell proliferation, said method comprising administering a therapeutically effective amount of the antibody-drug conjugate of claim 1, under conditions effective to treat a tumor or abnormal cell proliferation.

23-24. (canceled)

25. A method for stimulating an immune response in a subject, the method comprising administering a therapeutically effective amount of the antibody-drug conjugate of claim 1 under conditions effective to stimulate an immune response.

26-27. (canceled)

28. A method for inducing an anti-tumor immune response in a subject, the method comprising administering to a therapeutically effective amount of the antibody-drug conjugate of claim 1 under conditions effective to induce an anti-tumor immune response.

29-30. (canceled)
